SEQUENCE LISTING (64 sequences)

SEQ ID NO: 1 - DNA, 2969 bp, Homo sapiens
  source 1..2969: mol_type = genomic DNA
actgcaagctctgcctcccgggttcacgccattctcctgtgtcagcctcctgagtagctgggactacaggcgcccgccaccatgcccggctaattttttttgtatttttagtagagacggggtttcaccatgttagccaggatagtttcaatttccggacctcgtgatccgcccgcctcggcctcccaaagtgctgggattataggcgtgagccaccgcgcccagcccctttcatttttgatatggagttttgttcttgttgcccaggctggaatgcaatggcgcgatctcagctcactgcaacctctgcctcctgggttcaagcgattctcctggttcagcctcctgagtagctgggattacaggaatgcgccaccacacctggctacttttgtatttttagtagagatggggtttctccacgtgcatcaggctagtctcacactcccgacttcagatgatccacccgcctcggcctcccaaagtgctgggattacaggcgtgagccaccgtgcctggccaaagccttcttcactttcattttgtggctgctgttgtgaaatctgcagaacctcaagctgtttggactccatcttctaggttgaattagattacaggtaatctcatggtggaggtgtattctggatcgactcacttttgaaatgtttatgaaagaaattccttcacctcgggacttccacactggagctggcaatcagtatcaccttaatttacctatcacagtgaggcatctctgctctgaggaaaacaaaacaaaacaaaacaaaacaaaaaagaagagctaatggttctgttttgttttgttgttttagagacagggtcttgctctgtcgcccgggctggaatgcagtgacatcatcacagctcactgtagccttgatctcctgggctcaagcgatccttctgcctcagcctgagtagctgggatgggactacagatgcacgccaccacacatggctaatttttttattttttagagacgaggtcccatgtgagccacggtgccccaccttttaaagcaattaccgtaaggaagcaactgtctttagcaaatagaagatcataaacatccttctggagatcttgagatgttgactgtggtttatagggcaatgaaaatctacatacctgagtggggcagtgagacatgagtgccacctatgatccttccagtgagcaattctttctggtgccaggagcccaaaggaaactcagtcaaccagctgtagatgggcttgttccgagtttgttctagaagtcagaactgccagctctgaaagtaggtcacaagtgactggccatccctgggctgctcacaggaagtagctgcaaaggtcagggaaagtttttaatgactatacaggccagtgaatgacaggctttgaggagcttctcttcttggcatctgaaacaggcacatggaggagctgccttggagacagaatgacagctgtggcaatgtgagcctgattcctcagccccataaaatagatgcctttgcaaaaaacccatttatttttttttttttctgtttttgagagggagtctcgctctgtccccccaggcggagtgcagtggcgtgatctcggctcactgcaacttctgcctcccgggttcaagcgattctcctgcctcagctcccaagtagctgagattacaggcatccgccaccacgcctggccaattttgtattttagtagagacggggttttgccacattggccaggctgggaaaaagcccatttctatacagaggaatcatcttgcaggactcaaggaatgattaagaggaatctacagcattaactttagaaactcgatgcaatattcccaagaggagttaagattgggataagtaatctctgaattaatttcaaccttgagatgttgtgatcctatgaagtcaaagacatatttcttttttctttgtttgagatggagtcttgctctgtcacccaaattggagggcggtgatgtgatctcggcttactacgacctctgcctcctgggttcaagcgattctcagcttcagcctcctgagtagctgagattacaggtgcccgccaccatgcccggctaatttttgtatttttagtagagatgaggtttcactgtgttggccaggctggtcttgaattcttggccccaggtgatctgcccgcctgggcctcccaaagtgctgggattacaggagtgagcaccatgtccggtctagatacatatttctattaatacacatttgcagggattgtttgttactgaagttgacgccttgtgcttgagaatgctatttcactgacacaattgttatggtataaatgtgagagtatagttacaatctggacactattttttgcttgtcttattggcccagcaaccagcttgacactggggactatcaggctccaaataataaccaatgtctcactccaaacagacaggatactacggagccagggtcagcaaacattttctgtaaagggccagatagtaaatattttgggctttgtgggccctatggtctctgtcacaacgattcaactctgctgttgtaacaaaaaagcagtcagacaatatgtaaatgaatgggcatggctgtgttccagtaaaaccacaaaaacaggcagctagctttggtgtggggccctaagagcttggactttgaagccaggttcaaatcctggctttctcaccttctagctccgttaccttaaagaacttcattatcttccttgtattttagtcttttcatctctaaattagtgatagtacttaacagtgttgttataaagattaaacgagttaagtttgtaaagtacttaaacagtgcctaccaggtagtaagtgctatagcagagtgtgttagataatgaaacatcctgtgctgtggatatattattcccattacgatttggattcaaataaagaaatacatgacttagaagaa

SEQ ID NO: 2 - protein, 156 aa, Homo sapiens
MLARIVSISGPRDPPASASQSAGIIGVSHRAQPLSFLIWSFVLVAQAGMQWRDLSSLQPLPPGFKRFSWFSLLSSWDYRNAPPHLATFVFLVEMGFLHVHQASLTLPTSDDPPASASQSAGITGVSHRAWPKPSSLSFCGCCCEICRTSSCLDSIF

SEQ ID NO: 3 - DNA, 803 bp, Homo sapiens
  source 1..803: mol_type = genomic DNA
gagagatttcatctacgggaaaaagaccctctgaaaagaagtgtgaaatcatcaaaagagccattggaggggacaccagagagtcgtgtggctgaggtttcacatgatcccgccatgcttccagtccagaggagaaagctagacccactcctcaaaaagtataggcatcataagaaggccacaaggacaaaaagaaggaggaaggagaaaatggaggcccagtgctcccctgttcctccaacaccttcaacaccaccacaaagtgaagaagatgaggctgtagacaaaaagccaactctactcagtgcccaggaagatactcctgaccttctccatgaggacagattacagtatctgcaggaagagggttccggtgtgatgcatcaggaatgtcagatccagtcctgtgagctctcggtggctcagaagcccaggccctcttctcctgcagtgacatccttggcatcaccaccactctgctttggcagtttcctaagctgtgtctgccagaccttctcaaggtctaggaagcagaagcctcccagaagaaagggtaacaaccaggctgaggcaggaggtgatgctgaggtcctgagacctggcccggcaaaaccagagttgagcctcagcaaaacgtgtagccacttgaaatcgatagagctttcctttgtgtttagttttatagttctctctgtatgtcattgttcctcttagttgagtttttgcctgtcaaagagatagagagctgcttttatgctgagatcctcaagccaaaacccacagtaaacgcaaagtaaaacttt

SEQ ID NO: 4 - protein, 199 aa, Homo sapiens
MLPVQRRKLDPLLKKYRHHKKATRTKRRRKEKMEAQCSPVPPTPSTPPQSEEDEAVDKKPTLLSAQEDTPDLLHEDRLQYLQEEGSGVMHQECQIQSCELSVAQKPRPSSPAVTSLASPPLCFGSFLSCVCQTFSRSRKQKPPRRKGNNQAEAGGDAEVLRPGPAKPELSLSKTCSHLKSIELSFVFSFIVLSVCHCSS

SEQ ID NO: 5 - DNA, 25 bp, Homo sapiens
  source 1..25: mol_type = genomic DNA
ggccaaagccttcttcactttcatt

SEQ ID NO: 6 - DNA, 25 bp, synthetic construct
  source 1..25: mol_type = other DNA
  misc_feature 1..23: note = RNA
  misc_feature 24..25: note = DNA
ggccaaagccttcttcactttcatt

SEQ ID NO: 7 - RNA, 27 nt, synthetic construct
  source 1..27: mol_type = other RNA
aatgaaagtgaagaaggctttggccag

SEQ ID NO: 8 - DNA, 25 bp, Homo sapiens
  source 1..25: mol_type = genomic DNA
ctacttttgtatttttagtagagat

SEQ ID NO: 9 - DNA, 25 bp, synthetic construct
  source 1..25: mol_type = other DNA
  misc_feature 1..23: note = RNA
  misc_feature 24..25: note = DNA
ctacttttgtatttttagtagagat

SEQ ID NO: 10 - RNA, 27 nt, synthetic construct
  source 1..27: mol_type = other RNA
atctctactaaaaatacaaaagtagcc

SEQ ID NO: 11 - DNA, 25 bp, Homo sapiens
  source 1..25: mol_type = genomic DNA
cgatctcagctcactgcaacctctg

SEQ ID NO: 12 - DNA, 25 bp, synthetic construct
  source 1..25: mol_type = other DNA
  misc_feature 1..23: note = RNA
  misc_feature 24..25: note = DNA
cgatctcagctcactgcaacctctg

SEQ ID NO: 13 - RNA, 27 nt, synthetic construct
  source 1..27: mol_type = other RNA
cagaggttgcagtgagctgagatcgcg

SEQ ID NO: 14 - DNA, 25 bp, Homo sapiens
  source 1..25: mol_type = genomic DNA
ccatgaggacagattacagtatctg

SEQ ID NO: 15 - DNA, 25 bp, synthetic construct
  source 1..25: mol_type = other DNA
  misc_feature 1..23: note = RNA
  misc_feature 24..25: note = DNA
ccatgaggacagattacagtatctg

SEQ ID NO: 16 - RNA, 27 nt, synthetic construct
  source 1..27: mol_type = other RNA
cagatactgtaatctgtcctcatggag

SEQ ID NO: 17 - DNA, 25 bp, Homo sapiens
  source 1..25: mol_type = genomic DNA
catcaggaatgtcagatccagtcct

SEQ ID NO: 18 - DNA, 25 bp, synthetic construct
  source 1..25: mol_type = other DNA
  misc_feature 1..23: note = RNA
  misc_feature 24..25: note = DNA
catcaggaatgtcagatccagtcct

SEQ ID NO: 19 - RNA, 27 nt, synthetic construct
  source 1..27: mol_type = other RNA
aggactggatctgacattcctgatgca

SEQ ID NO: 20 - DNA, 25 bp, Homo sapiens
  source 1..25: mol_type = genomic DNA
acttgaaatcgatagagctttcctt

SEQ ID NO: 21 - DNA, 25 bp, synthetic construct
  source 1..25: mol_type = other DNA
  misc_feature 1..23: note = RNA
  misc_feature 24..25: note = DNA
acttgaaatcgatagagctttcctt

SEQ ID NO: 22 - RNA, 27 nt, synthetic construct
  source 1..27: mol_type = other RNA
aaggaaagctctatcgatttcaagtgg

SEQ ID NO: 23 - DNA, 1431 bp, Homo sapiens
  source 1..1431: mol_type = genomic DNA
atggacttttccatctgcatcagtaacatcaccccagcagatgccggcacctactactgtgtgaagttccagaaagggagccctgacgtggagttgaagtctggagcaggcactgagctgtctgtgcgtgccaaaccctctgcccccgtggtatcgggccccgcagcgagggccacacctgaccacacagtgagcttcacctgcgagtctcatggcttctcacccagagacatcagcctgaaatggttcaaaaatgggaatcagctctcagacttccagaccaacgtggaccccgcaagagagagcgtgtcctacagcatccacagcacagccaatgtggtgctgacccgcggggacattcactctcaagtcatctgcgaggtggcccacgtcaccttgcggggggactcttttcgtgggactgccaacttgtctgagactatccaagttccacccaccttggaggttactcaacagcccatgagggcagagaaccaggtgaatatcacctgccaggtgacgaaattctacccccagagactacagttgacctggttggagaacggcaatgtgtcccggacagaaacggcctcaactcttacagagaacaaggatggcacctacaactggatgagctggctcctggtgaatgtatctgcccacagggatgatgtgaagctcacctgccaggtggagcatgacgggcagtcagcggtcagcaaaagccatgacctgaaggtctcagcccacctgaaggagcagagctcaaataccgccgctgagaacactggacctaatgaacagaacatctatattgtggtgggcgtggtgtgcaccttgctggtggccctactgatggaggctctctacctcgtccgaatcagacagaagaaagcccagggctccacttcttctacaaggttgcatgaacccgagaagaatgccagaaaaataacccaggacacaaatgatatcacatatgcggacctgaacctgcccaaggggaagaagcctgctccccgggccgcggagcccaacaaccacacagagtatgccagcattcagaccagcctgcagcctgcgtcggaggacaccctcacctatgctgacctggacatggtgcacctcaaccggacccccaagcagctggcccccaagcccgagctgtccttctcagagtatgccagcatccaggtcccgaggaagtgaatgggaccgtggtttgctctagcacccatctccatgctcttccttgtcccacaaggagccgccatgatgagcacagccagcccagttcccggagggctggggcggtgcaggctctgggacccaggggccagggtggctcttctctccccacccctccttggctctccagcagccacggccccctctccccacattgccacacacctggaggctgacgttgcc

SEQ ID NO: 24 - DNA, 686 bp, Homo sapiens
  source 1..686: mol_type = genomic DNA
atggggattccaggcctggagcacctgcacatctggatctccatccccttctcagcatatacactggccctgcttggaaactgcaccctccttctcatcatccaggctgatgcagccctccatgagcccatatacctctttctggccatgttggcagccatcgacctggtcctttcctcctcagcattgcccaaaatgcttgccatattctggttcagggatcgggagatcaacttttttgcctgtctggtccagatgttcttccttcactccttctccatcatggagtcagcagtgctgctggccatggcctttgaccgctatgtggccatctgcaagccactgcactacaccacggtcctgactgggtccctcatcaccaagattggcatggctgctgtggcccgggctgtgacactaatgactccactccccttcctgctgagatgtttccactactgccgaggcccagtgattgcccgctgctactgtgaacacatggctgtggtcaggctggctgtgggaacactagcttcaacaatatctatggcattgctgtggccatgtttattggagtgttggatctattctttatcatcctatcttatatctttatccttcaggcagttctacaactctcctctcaggaggcccgctacaaagcatttgggacatgtgtctctcac

SEQ ID NO: 25 - DNA, 25 bp, Homo sapiens
  source 1..25: mol_type = genomic DNA
cagcaaaagccatgacctgaaggtc

SEQ ID NO: 26 - DNA, 25 bp, synthetic construct
  source 1..25: mol_type = other DNA
  misc_feature 1..23: note = RNA
  misc_feature 24..25: note = DNA
acattcactctcaagtcatctgcga

SEQ ID NO: 27 - RNA, 27 nt, synthetic construct
  source 1..27: mol_type = other RNA
tcgcagatgacttgagagtgaatgtcc

SEQ ID NO: 28 - DNA, 25 bp, Homo sapiens
  source 1..25: mol_type = genomic DNA
aacttgtctgagactatccaagttc

SEQ ID NO: 29 - DNA, 25 bp, synthetic construct
  source 1..25: mol_type = other DNA
  misc_feature 1..23: note = RNA
  misc_feature 24..25: note = DNA
cagcaaaagccatgacctgaaggtc

SEQ ID NO: 30 - RNA, 27 nt, synthetic construct
  source 1..27: mol_type = other RNA
gaccttcaggtcatggcttttgctgac

SEQ ID NO: 31 - DNA, 25 bp, Homo sapiens
  source 1..25: mol_type = genomic DNA
acattcactctcaagtcatctgcga

SEQ ID NO: 32 - DNA, 25 bp, synthetic construct
  source 1..25: mol_type = other DNA
  misc_feature 1..23: note = RNA
  misc_feature 24..25: note = DNA
aacttgtctgagactatccaagttc

SEQ ID NO: 33 - RNA, 27 nt, synthetic construct
  source 1..27: mol_type = other RNA
gaacttggatagtctcagacaagttgg

SEQ ID NO: 34 - DNA, 25 bp, Homo sapiens
  source 1..25: mol_type = genomic DNA
ctacaaagcatttgggacatgtgtc

SEQ ID NO: 35 - DNA, 25 bp, synthetic construct
  source 1..25: mol_type = other DNA
  misc_feature 1..23: note = RNA
  misc_feature 24..25: note = DNA
ctacaaagcatttgggacatgtgtc

SEQ ID NO: 36 - RNA, 27 nt, synthetic construct
  source 1..27: mol_type = other RNA
gacacatgtcccaaatgctttgtagcg

SEQ ID NO: 37 - DNA, 25 bp, Homo sapiens
  source 1..25: mol_type = genomic DNA
cttcaacaatatctatggcattgct

SEQ ID NO: 38 - DNA, 25 bp, synthetic construct
  source 1..25: mol_type = other DNA
  misc_feature 1..23: note = RNA
  misc_feature 24..25: note = DNA
cttcaacaatatctatggcattgct

SEQ ID NO: 39 - RNA, 27 nt, synthetic construct
  source 1..27: mol_type = other RNA
agcaatgccatagatattgttgaagct

SEQ ID NO: 40 - DNA, 25 bp, Homo sapiens
  source 1..25: mol_type = genomic DNA
ccttcctgctgagatgtttccacta

SEQ ID NO: 41 - DNA, 25 bp, synthetic construct
  source 1..25: mol_type = other DNA
  misc_feature 1..23: note = RNA
  misc_feature 24..25: note = DNA
ccttcctgctgagatgtttccacta

SEQ ID NO: 42 - RNA, 27 nt, synthetic construct
  source 1..27: mol_type = other RNA
tagtggaaacatctcagcaggaagggg

SEQ ID NO: 43 - DNA, 18 bp, synthetic construct
  source 1..18: mol_type = other DNA
cgcccagcccctttcatt

SEQ ID NO: 44 - DNA, 17 bp, synthetic construct
  source 1..17: mol_type = other DNA
cgcgccattgcattcca

SEQ ID NO: 45 - DNA, 23 bp, synthetic construct
  source 1..23: mol_type = other DNA
acaagaacaaaactccatatcaa

SEQ ID NO: 46 - DNA, 25 bp, Homo sapiens
  source 1..25: mol_type = genomic DNA
tttgatatggagttttgttcttgtt

SEQ ID NO: 47 - DNA, 23 bp, synthetic construct
  source 1..23: mol_type = other DNA
cctgccaggtgacgaaattctac

SEQ ID NO: 48 - DNA, 19 bp, synthetic construct
  source 1..19: mol_type = other DNA
cgggacacattgccgttct

SEQ ID NO: 49 - DNA, 18 bp, synthetic construct
  source 1..18: mol_type = other DNA
ccaaccaggtcaactgta

SEQ ID NO: 50 - DNA, 25 bp, Homo sapiens
  source 1..25: mol_type = genomic DNA
ctacagttgacctggttggagaacg

SEQ ID NO: 51 - DNA, 22 bp, synthetic construct
  source 1..22: mol_type = other DNA
ctcagtgcccaggaagatactc

SEQ ID NO: 52 - DNA, 20 bp, synthetic construct
  source 1..20: mol_type = other DNA
catcacaccggaaccctctt

SEQ ID NO: 53 - DNA, 17 bp, synthetic construct
  source 1..17: mol_type = other DNA
ccatgaggacagattac

SEQ ID NO: 54 - DNA, 25 bp, Homo sapiens
  source 1..25: mol_type = genomic DNA
ctcagtgcccaggaagatactcctg

SEQ ID NO: 55 - DNA, 20 bp, synthetic construct
  source 1..20: mol_type = other DNA
cccgggctgtgacactaatg

SEQ ID NO: 56 - DNA, 22 bp, synthetic construct
  source 1..22: mol_type = other DNA
cggcagtagtggaaacatctca

SEQ ID NO: 57 - DNA, 18 bp, synthetic construct
  source 1..18: mol_type = other DNA
actccactccccttcctg

SEQ ID NO: 58 - DNA, 25 bp, Homo sapiens
  source 1..25: mol_type = genomic DNA
cccgggctgtgacactaatgactcc

SEQ ID NO: 59 - DNA, 19 bp, synthetic construct
  source 1..19: mol_type = other DNA
acatcgctcagacaccatg

SEQ ID NO: 60 - DNA, 22 bp, synthetic construct
  source 1..22: mol_type = other DNA
tgtagttgaggtcaatgaaggg

SEQ ID NO: 61 - DNA, 25 bp, synthetic construct
  source 1..25: mol_type = other DNA
aaggtcggagtcaacggatttggtc

SEQ ID NO: 62 - DNA, 22 bp, synthetic construct
  source 1..22: mol_type = other DNA
ttgttgtaggatatgcccttga

SEQ ID NO: 63 - DNA, 21 bp, synthetic construct
  source 1..21: mol_type = other DNA
gcgatgtcaataggactccag

SEQ ID NO: 64 - DNA, 31 bp, synthetic construct
  source 1..31: mol_type = other DNA
agcctaagatgagagttcaagttgagtttgg

(19) United States
(12) Patent Application Publication
(10) Pub. No.: US 2026/0152745 A1
(43) Pub. Date: Jun. 4, 2026

(54) COMPOSITIONS AND METHODS FOR TREATMENT OF CANCER

(71) Applicant: NonExomics, Inc., Acton, MA (US)

(72) Inventors: Ruchi CHAUHAN, Acton, MA (US); Sudhakaran PRABAKARAN, Acton, MA (US)

(21) Appl. No.: 18/875,017

(22) PCT Filed: Jun. 15, 2023
(86) PCT No.: PCT/US2023/025450

§ 371 Date: Dec. 13, 2024

Related U.S. Application Data
(60) Provisional application No. 63/352,520, filed on Jun. 15, 2022.

(51) Int. Cl. C12N 15/113; A61P 35/00

(52) U.S. Cl. CPC C12N 15/1135; A61P 35/00; C12N 2310/14

(57) ABSTRACT

The present application features methods of treating cancer by administering a polynucleotide that reduces expression of a dysregulated novel open reading frame (nORF) in which increased expression of the dysregulated nORF is associated with the cancer.

### BACKGROUND OF THE INVENTION

Many cancers are caused by genetic dysregulation. However, it is unclear how cancer pathology ensues when the genetic dysregulation does not occur in a canonical gene. Furthermore, providing an effective therapeutic remains a challenging endeavor for many cancers. Accordingly, new methods of treatment are needed to better treat these cancers.

### SEQUENCE LISTING

The present application includes a Sequence Listing filed in ST.26 format. The Sequence Listing, created on Feb. 18, 2026, is entitled “5275-0109US2-SequenceListing.xml” and is 72,588 bytes in size. The information in the electronic format of the Sequence Listing is part of the present application and is incorporated herein by reference in its entirety.

### SUMMARY OF THE INVENTION

In one aspect, the invention features a method of treating cancer in a subject by administering to the subject a polynucleotide that reduces expression of a gene ENST00000455557.2 having the sequence of SEQ ID NO: 1 to treat the cancer.

In some embodiments, the cancer is head and neck cancer, bladder urothelial carcinoma, breast invasive carcinoma, cervical cancer, endocervical cancer, colon adenocarcinoma, esophageal carcinoma, kidney clear cell carcinoma, kidney papillary cell carcinoma, lung adenocarcinoma, lung squamous cell carcinoma, ovarian serous cystadenocarcinoma, pancreatic adenocarcinoma, rectum adenocarcinoma, skin cutaneous melanoma, stomach adenocarcinoma, uterine carcinosarcoma, or uterine corpus endometrioid carcinoma. In some embodiments, the cancer is head and neck cancer.

In some embodiments, the polynucleotide targets any one or more (e.g., 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or more) of nucleotides 130-597 of SEQ ID NO: 1. In some embodiments, the polynucleotide targets one or more of nucleotides 130-180, 180-230, 230-280, 280-330, 330-380, 380-430, 430-480, 480-530, 530-580, or 580-597 of SEQ ID NO: 1. For example, the polynucleotide may target one or more of nucleotides 130-150, 150-170, 170-190, 190-210, 210-230, 230-250, 250-270, 270-290, 290-310, 310-330, 330-350, 350-370, 370-390, 390-410, 410-430, 430-450, 450-470, 470-490, 490-510, 510-530, 530-550, 550-570, 570-590, or 590-597 of SEQ ID NO: 1.

In some embodiments, the polynucleotide has a nucleobase sequence including a portion of at least 10 (e.g., at least 11, 12, 13, 14, 15, 16, 17, 18, 19, 20, 25, 30, 35, 40, 45, 50, or more) contiguous nucleobases having at least 80% (e.g., at least 85%, 90%, 95%, 97%, 99%, or 100%) complementarity to an equal length portion of nucleotides 130-597 of SEQ ID NO: 1.

In some embodiments, the oligonucleotide includes a region complementary to at least 17 contiguous nucleotides of SEQ ID NO: 1.

In some embodiments, the oligonucleotide includes a region complementary to at least 19 contiguous nucleotides of SEQ ID NO: 1.

In some embodiments, the method reduces expression (e.g., by at least 10%, 20%, 30%, 40%, 50%, 60%, 70%, 80%, 90%, 95%, 97%, 99%, or more) of a polypeptide having the sequence of SEQ ID NO: 2.

In some embodiments, the polynucleotide has at least 80% (e.g., at least 85%, 90%, 95%, 97%, 99%, or 100%) sequence identity to any one of SEQ ID NOs: 5-13. In some embodiments, the polynucleotide includes the sequence of any one of SEQ ID NOs: 5-13.

In another aspect, the invention features a method of treating cancer in a subject by administering to the subject a polynucleotide that reduces expression of a gene ENST00000383686.3 having the sequence of SEQ ID NO: 3 to treat the cancer.

In some embodiments, the cancer is brain lower grade glioma, breast invasive carcinoma, diffuse large B cell lymphoma, esophageal carcinoma, kidney clear cell carcinoma, kidney papillary cell carcinoma, liver hepatocellular carcinoma, lung adenocarcinoma, lung squamous cell carcinoma, ovarian serous cystadenocarcinoma, pancreatic adenocarcinoma, prostate adenocarcinoma, skin cutaneous melanoma, stomach adenocarcinoma, testicular germ cell tumor, thymoma, thyroid carcinoma, uterine carcinosarcoma, or uterine corpus endometrioid carcinoma. In some embodiments, the cancer is breast invasive carcinoma.

In some embodiments, the polynucleotide targets any one or more (e.g., 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or more) of nucleotides 115-711 of SEQ ID NO: 3. In some embodiments, the polynucleotide targets one or more of nucleotides 115-165, 165-215, 215-265, 265-315, 315-365, 365-415, 415-465, 465-515, 515-565, 565-615, 615-665, or 665-711 of SEQ ID NO: 3. For example, the polynucleotide may target one or more of nucleotides 115-135, 135-155, 155-175, 175-195, 195-215, 215-235, 235-255, 255-275, 275-295, 295-315, 315-335, 335-355, 355-375, 375-395, 395-415, 415-435, 435-455, 455-475, 475-495, 495-515, 515-535, 535-555, 555-575, 575-595, 595-615, 615-635, 635-655, 655-675, 675-695, or 695-711 of SEQ ID NO: 3.

In some embodiments, the polynucleotide has a nucleobase sequence including a portion of at least 10 (e.g., at least 11, 12, 13, 14, 15, 16, 17, 18, 19, 20, 25, 30, 35, 40, 45, 50, or more) contiguous nucleobases having at least 80% (e.g., at least 85%, 90%, 95%, 97%, 99%, or 100%) complementarity to an equal length portion of nucleotides 115-711 of SEQ ID NO: 3.

In some embodiments, the oligonucleotide includes a region complementary to at least 17 contiguous nucleotides of SEQ ID NO: 3.

In some embodiments, the oligonucleotide includes a region complementary to at least 19 contiguous nucleotides of SEQ ID NO: 3.

In some embodiments, the method reduces expression (e.g., by at least 10%, 20%, 30%, 40%, 50%, 60%, 70%, 80%, 90%, 95%, 97%, 99%, or more) of a polypeptide having the sequence of SEQ ID NO: 4.

In some embodiments, the polynucleotide has at least 80% (e.g., at least 85%, 90%, 95%, 97%, 99%, or 100%) sequence identity to any one of SEQ ID NOs: 14-22. In some embodiments, the polynucleotide includes the sequence of any one of SEQ ID NOs: 14-22.

In some embodiments, the polynucleotide includes a miRNA, an antisense polynucleotide, an shRNA, or an siRNA. In some embodiments, the polynucleotide is a miRNA. In some embodiments the polynucleotide is an antisense polynucleotide (e.g., antisense oligonucleotide). In some embodiments, the polynucleotide is an shRNA. In some embodiments, the polynucleotide is an siRNA.

In some embodiments, the polynucleotide consists of 12 to 80 (e.g., 12 to 40, 16 to 30, e.g., 18 to 22, e.g., 12, 13, 14, 15, 16, 17, 18, 19, 20, 21, 22, 23, 24, 25, 26, 27, 28, 29, 30, 31, 32, 33, 34, 35, 36, 37, 38, 39, or 40) nucleobases. In some embodiments, the polynucleotide consists of 12 to 40 nucleobases. In some embodiments, the polynucleotide consists of 16 to 30 nucleobases. In some embodiments, the polynucleotide consists of 18 to 22 nucleobases.

In some embodiments, the polynucleotide includes at least one alternative internucleoside linkage. The at least one alternative internucleoside linkage may be, e.g., a phosphorothioate, a 2′-alkoxy, or an alkyl phosphate internucleoside linkage.

In some embodiments, the polynucleotide includes at least one alternative nucleobase. The alternative nucleobase may be, e.g., 5′-methylcytosine, pseudouridine, or 5-methoxyuridine. In some embodiments, the polynucleotide includes at least one alternative sugar moiety. The alternative sugar moiety may be, e.g., 2′-OMethyl modified sugar moiety or a bicyclic sugar moiety.

In some embodiments, the polynucleotide is encoded by a vector, such as a viral vector. The viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an adeno-associated virus (AAV) vector.

In some embodiments, the viral vector is a Retroviridae family viral vector (e.g., a lentiviral vector, an alpharetroviral vector, or a gammaretroviral vector). The Retroviridae family viral vector may include one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be a lentiviral vector.

In some embodiments, the pseudotyped viral vector includes one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV). In some embodiments, the pseudotyped viral vector includes a VSV-G envelope protein.

In another aspect, the invention features a polynucleotide that targets SEQ ID NO: 1, such as one or more of nucleotides 130-597 of SEQ ID NO: 1.

In some embodiments, the polynucleotide targets any one or more (e.g., 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or more) of nucleotides 130-597 of SEQ ID NO: 1. In some embodiments, the polynucleotide targets one or more of nucleotides 130-180, 180-230, 230-280, 280-330, 330-380, 380-430, 430-480, 480-530, 530-580, or 580-597 of SEQ ID NO: 1. For example, the polynucleotide may target one or more of nucleotides 130-150, 150-170, 170-190, 190-210, 210-230, 230-250, 250-270, 270-290, 290-310, 310-330, 330-350, 350-370, 370-390, 390-410, 410-430, 430-450, 450-470, 470-490, 490-510, 510-530, 530-550, 550-570, 570-590, or 590-597 of SEQ ID NO: 1.

In some embodiments, the polynucleotide has a nucleobase sequence including a portion of at least 10 (e.g., at least 11, 12, 13, 14, 15, 16, 17, 18, 19, 20, 25, 30, 35, 40, 45, 50, or more) contiguous nucleobases having at least 80% (e.g., at least 85%, 90%, 95%, 97%, 99%, or 100%) complementarity to an equal length portion of nucleotides 130-597 of SEQ ID NO: 1.

In some embodiments, the oligonucleotide includes a region complementary to at least 17 contiguous nucleotides of SEQ ID NO: 1.

In some embodiments, the oligonucleotide includes a region complementary to at least 19 contiguous nucleotides of SEQ ID NO: 1.

In some embodiments, the polynucleotide reduces expression (e.g., by at least 10%, 20%, 30%, 40%, 50%, 60%, 70%, 80%, 90%, 95%, 97%, 99%, or more) of a polypeptide having the sequence of SEQ ID NO: 2.

In some embodiments, the polynucleotide has at least 80% (e.g., at least 85%, 90%, 95%, 97%, 99%, or 100%) sequence identity to any one of SEQ ID NOs: 5-13. In some embodiments, the polynucleotide includes the sequence of any one of SEQ ID NOs: 5-13.

In another aspect, the invention features a polynucleotide that targets SEQ ID NO: 3, such as one or more of nucleotides 115-711 of SEQ ID NO: 3.

In some embodiments, the polynucleotide targets any one or more (e.g., 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, or more) of nucleotides 115-711 of SEQ ID NO: 3. In some embodiments, the polynucleotide targets one or more of nucleotides 115-165, 165-215, 215-265, 265-315, 315-365, 365-415, 415-465, 465-515, 515-565, 565-615, 615-665, or 665-711 of SEQ ID NO: 3. For example, the polynucleotide may target one or more of nucleotides 115-135, 135-155, 155-175, 175-195, 195-215, 215-235, 235-255, 255-275, 275-295, 295-315, 315-335, 335-355, 355-375, 375-395, 395-415, 415-435, 435-455, 455-475, 475-495, 495-515, 515-535, 535-555, 555-575, 575-595, 595-615, 615-635, 635-655, 655-675, 675-695, or 695-711 of SEQ ID NO: 3.

In some embodiments, the polynucleotide has a nucleobase sequence including a portion of at least 10 (e.g., at least 11, 12, 13, 14, 15, 16, 17, 18, 19, 20, 25, 30, 35, 40, 45, 50, or more) contiguous nucleobases having at least 80% (e.g., at least 85%, 90%, 95%, 97%, 99%, or 100%) complementarity to an equal length portion of nucleotides 115-711 of SEQ ID NO: 3.

In some embodiments, the oligonucleotide includes a region complementary to at least 17 contiguous nucleotides of SEQ ID NO: 3.

In some embodiments, the oligonucleotide includes a region complementary to at least 19 contiguous nucleotides of SEQ ID NO: 3.

In some embodiments, the polynucleotide reduces expression (e.g., by at least 10%, 20%, 30%, 40%, 50%, 60%, 70%, 80%, 90%, 95%, 97%, 99%, or more) of a polypeptide having the sequence of SEQ ID NO: 4.

In some embodiments, the polynucleotide has at least 80% (e.g., at least 85%, 90%, 95%, 97%, 99%, or 100%) sequence identity to any one of SEQ ID NOs: 14-22. In some embodiments, the polynucleotide includes the sequence of any one of SEQ ID NOs: 14-22.

In some embodiments, the polynucleotide includes a miRNA, an antisense polynucleotide, an shRNA, or an siRNA. In some embodiments, the polynucleotide is a miRNA. In some embodiments the polynucleotide is an antisense polynucleotide (e.g., antisense oligonucleotide). In some embodiments, the polynucleotide is an shRNA. In some embodiments, the polynucleotide is an siRNA.

In some embodiments, the polynucleotide consists of 12 to 80 (e.g., 12 to 40, 16 to 30, e.g., 18 to 22, e.g., 12, 13, 14, 15, 16, 17, 18, 19, 20, 21, 22, 23, 24, 25, 26, 27, 28, 29, 30, 31, 32, 33, 34, 35, 36, 37, 38, 39, or 40) nucleobases. In some embodiments, the polynucleotide consists of 12 to 40 nucleobases. In some embodiments, the polynucleotide consists of 16 to 30 nucleobases. In some embodiments, the polynucleotide consists of 18 to 22 nucleobases.

In some embodiments, the polynucleotide includes at least one alternative internucleoside linkage. The at least one alternative internucleoside linkage may be, e.g., a phosphorothioate, a 2′-alkoxy, or an alkyl phosphate internucleoside linkage.

In some embodiments, the polynucleotide includes at least one alternative nucleobase. The alternative nucleobase may be, e.g., 5′-methylcytosine, pseudouridine, or 5-methoxyuridine.

In some embodiments, the polynucleotide includes at least one alternative sugar moiety. The alternative sugar moiety may be, e.g., 2′-OMethyl modified sugar moiety or a bicyclic sugar moiety.

In some embodiments, the polynucleotide is encoded by a vector, such as a viral vector. The viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an adeno-associated virus (AAV) vector.

In some embodiments, the viral vector is a Retroviridae family viral vector (e.g., a lentiviral vector, an alpharetroviral vector, or a gammaretroviral vector). The Retroviridae family viral vector may include one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be a lentiviral vector.

In some embodiments, the pseudotyped viral vector includes one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV). In some embodiments, the pseudotyped viral vector includes a VSV-G envelope protein.

### Definitions

As used herein, a “novel open reading frame” or “nORF” refers to an open reading frame that is transcribed in a cell and consists of a sequence that is distinct from a canonical open reading frame (cORF) transcribed from a gene. The nORF may be present in (i) an overlapping region of the cORF in an alternate reading frame, (ii) a 5′ untranslated region (UTR) of the cORF, (iii) a 3′ UTR of the cORF, (iv) an intronic region of the cORF, (v) an intergenic region of the cORF, or (vi) a region not associated with the cORF or the gene. The nORF may be any unannotated genetic sequence that is transcribed in a cell.

As used herein, a “canonical open reading frame” or “cORF” refers to an open reading frame that is transcribed in a cell and its associated genetic elements, including the 5′ UTR, the 3′ UTR, the intronic regions, the exonic regions, and the intergenic regions flanking the gene including the cORF. A cORF includes either the primary open reading frame that is expressed from a gene, the most abundantly expressed open reading frame expressed from a gene, or an ORF that is annotated in a publicly available database as the primary and/or most abundantly expressed open reading frame from a gene.

As used herein, “target sequence” refers to a contiguous portion of the nucleotide sequence of an mRNA molecule formed during the transcription of a nORF gene, including mRNA that is a product of RNA processing of a primary transcription product. In one embodiment, the target portion of the sequence will be at least long enough to serve as a substrate for oligonucleotide-directed (e.g., antisense oligonucleotide (ASO)-directed) cleavage at or near that portion of the nucleotide sequence of an mRNA molecule formed during the transcription of a nORF gene. The target sequence may be, for example, from about 9-36 nucleotides in length, e.g., about 15-30 nucleotides in length, e.g., about 18-22 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

“G,” “C,” “A,” “T,” and “U” each generally stand for a naturally occurring nucleotide that contains guanine, cytosine, adenine, thymidine, and uracil as a base, respectively. However, it will be understood that the term “nucleotide” can also refer to an alternative nucleotide, as further detailed below, or a surrogate replacement moiety. The skilled person is well aware that guanine, cytosine, adenine, and uracil can be replaced by other moieties without substantially altering the base pairing properties of an oligonucleotide including a nucleotide bearing such replacement moiety. For example, without limitation, a nucleotide including inosine as its base can base pair with nucleotides containing adenine, cytosine, or uracil. Hence, nucleotides containing uracil, guanine, or adenine can be replaced in the nucleotide sequences of oligonucleotides featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “nucleobase” and “base” include the purine (e.g., adenine and guanine) and pyrimidine (e.g., uracil, thymine, and cytosine) moiety present in nucleosides and nucleotides which form hydrogen bonds in nucleic acid hybridization. In the context of the present invention, the term nucleobase also encompasses alternative nucleobases which may differ from naturally occurring nucleobases but are functional during nucleic acid hybridization. In this context “nucleobase” refers to both naturally occurring nucleobases such as adenine, guanine, cytosine, thymidine, uracil, xanthine, and hypoxanthine, as well as alternative nucleobases. Such variants are for example described in Hirao et al (2012) Accounts of Chemical Research vol 45, page 2055 and Bergstrom (2009) Current Protocols in Nucleic Acid Chemistry Suppl. 37 1.4.1.

The term “nucleoside” refers to a monomeric unit of an oligonucleotide or a polynucleotide having a nucleobase and a sugar moiety. A nucleoside may include those that are naturally occurring as well as alternative nucleosides, such as those described herein. The nucleobase of a nucleoside may be a naturally occurring nucleobase or an alternative nucleobase. Similarly, the sugar moiety of a nucleoside may be a naturally occurring sugar or an alternative sugar.

The term “alternative nucleoside” refers to a nucleoside having an alternative sugar or an alternative nucleobase, such as those described herein.

In a some embodiments the nucleobase moiety is modified by changing the purine or pyrimidine into a modified purine or pyrimidine, such as substituted purine or substituted pyrimidine, such as an “alternative nucleobase” selected from isocytosine, pseudoisocytosine, 5-methyl cytosine, 5-thiozolo-cytosine, 5-propynyl-cytosine, 5-propynyl-uridine, 5-bromouridine 5-thiazolo-uridine, 2-thio-uridine, pseudouridine, 1-methylpseudouridine, 5-methoxyuridine, 2′-thio-thymine, inosine, diaminopurine, 6-aminopurine, 2-aminopurine, 2,6-diaminopurine, and 2-chloro-6-aminopurine.

The nucleobase moieties may be indicated by the letter code for each corresponding nucleobase, e.g., A, T, G, C, or U, wherein each letter may optionally include alternative nucleobases of equivalent function. In some embodiments, e.g., for gapmers, 5-methyl cytosine LNA nucleosides may be used.

A “sugar” or “sugar moiety” includes naturally occurring sugars having a furanose ring. A sugar also includes an “alternative sugar,” defined as a structure that is capable of replacing the furanose ring of a nucleoside. In certain embodiments, alternative sugars are non-furanose (or 4′-substituted furanose) rings or ring systems or open systems. Such structures include simple changes relative to the natural furanose ring, such as a six-membered ring, or may be more complicated as is the case with the non-ring system used in peptide nucleic acid. Alternative sugars may also include sugar surrogates wherein the furanose ring has been replaced with another ring system such as, for example, a morpholino or hexitol ring system. Sugar moieties useful in the preparation of oligonucleotides having motifs include, without limitation, β-D-ribose, β-D-2′-deoxyribose, substituted sugars (such as 2′,5′ and bis substituted sugars), 4′-S-sugars (such as 4′-S-ribose, 4′-S-2′-deoxyribose and 4′-S-2′-substituted ribose), bicyclic alternative sugars (such as the 2′-O—CH₂-4′ or 2′-O—(CH₂)₂-4′ bridged ribose derived bicyclic sugars) and sugar surrogates (such as when the ribose ring has been replaced with a morpholino or a hexitol ring system). The type of heterocyclic base and internucleoside linkage used at each position is variable and is not a factor in determining the motif. In most nucleosides having an alternative sugar moiety, the heterocyclic nucleobase is generally maintained to permit hybridization.

A “nucleotide,” as used herein, refers to a monomeric unit of an oligonucleotide or polynucleotide that includes a nucleoside and an internucleosidic linkage. The internucleosidic linkage may or may not include a phosphate linkage. Similarly, “linked nucleosides” may or may not be linked by phosphate linkages. Many “alternative internucleosidic linkages” are known in the art, including, but not limited to, phosphate, phosphorothioate, and boronophosphate linkages. Alternative nucleosides include bicyclic nucleosides (BNAs) (e.g., locked nucleosides (LNAs) and constrained ethyl (cEt) nucleosides), peptide nucleosides (PNAs), phosphotriesters, phosphorothionates, phosphoramidates, and other variants of the phosphate backbone of native nucleoside, including those described herein.

An “alternative nucleotide,” as used herein, refers to a nucleotide having an alternative nucleoside or an alternative sugar, and an internucleoside linkage, which may include alternative nucleoside linkages.

The terms “oligonucleotide” and “polynucleotide” as used herein are defined as it is generally understood by the skilled person as a molecule including two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention may be man-made, is chemically synthesized, and is typically purified or isolated. Oligonucleotide is also intended to include (i) compounds that have one or more furanose moieties that are replaced by furanose derivatives or by any structure, cyclic or acyclic, that may be used as a point of covalent attachment for the base moiety, (ii) compounds that have one or more phosphodiester linkages that are either modified, as in the case of phosphoramidate or phosphorothioate linkages, or completely replaced by a suitable linking moiety as in the case of formacetal or riboacetal linkages, and/or (iii) compounds that have one or more linked furanose-phosphodiester linkage moieties replaced by any structure, cyclic or acyclic, that may be used as a point of covalent attachment for the base moiety. The oligonucleotide of the invention may include one or more alternative nucleosides or nucleotides (e.g., including those described herein). It is also understood that oligonucleotide includes compositions lacking a sugar moiety or nucleobase but is still capable of forming a pairing with or hybridizing to a target sequence.

“Oligonucleotide” refers to a short polynucleotide (e.g., of 100 or fewer linked nucleosides).

“Chimeric” oligonucleotides or “chimeras,” in the context of this invention, are oligonucleotides which contain two or more chemically distinct regions, each made up of at least one monomer unit, i.e., a nucleotide or nucleoside in the case of an oligonucleotide. Chimeric oligonucleotides also include “gapmers.”

The term “gapmer” as used herein refers to an oligonucleotide which comprises a region of RNase H recruiting oligonucleotides (gap) which is flanked 5′ and 3′ by regions which comprise one or more affinity enhancing alternative nucleosides (wings or flanks). Various gapmer designs are described herein. Headmers and tailmers are oligonucleotides capable of recruiting RNase H where one of the wings is missing, i.e., only one of the ends of the oligonucleotide comprises affinity enhancing alternative nucleosides. For headmers the 3′ wing is missing (i.e., the 5′ wing comprises affinity enhancing alternative nucleosides) and for tailmers the 5′ wing is missing (i.e., the 3′ wing comprises affinity enhancing alternative nucleosides). A “mixed wing gapmer” refers to a gapmer wherein the wing regions comprise at least one alternative nucleoside, such as at least one DNA nucleoside or at least one 2′ substituted alternative nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-F-ANA nucleoside(s), or bicyclic nucleosides (e.g., locked nucleosides or constrained ethyl (cEt) nucleosides). In some embodiments the mixed wing gapmer has one wing which comprises alternative nucleosides (e.g., 5′ or 3′) and the other wing (3′ or 5′ respectfully) comprises 2′ substituted alternative nucleoside(s).

The term “linker” or “linking group” is a connection between two atoms that links one chemical group or segment of interest to another chemical group or segment of interest via one or more covalent bonds. Conjugate moieties can be attached to the oligonucleotide directly or through a linking moiety (e.g., linker or tether). Linkers serve to covalently connect a third region, e.g., a conjugate moiety to an oligonucleotide (e.g., the termini of region A or C). In some embodiments of the invention the conjugate or oligonucleotide conjugate of the invention may optionally, comprise a linker region which is positioned between the oligonucleotide and the conjugate moiety. In some embodiments, the linker between the conjugate and oligonucleotide is biocleavable. Phosphodiester containing biocleavable linkers are described in more detail in WO 2014/076195 (herein incorporated by reference).

As used herein, and unless otherwise indicated, the term “complementary,” when used to describe a first nucleotide or nucleoside sequence in relation to a second nucleotide or nucleoside sequence, refers to the ability of an oligonucleotide or polynucleotide comprising the first nucleotide or nucleoside sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides or nucleosides.

“Complementary” sequences, as used herein, can also include, or be formed entirely from, non-Watson-Crick base pairs and/or base pairs formed from non-natural and alternative nucleotides or nucleosides, in so far as the above requirements with respect to their ability to hybridize are fulfilled. Such non-Watson-Crick base pairs include, but are not limited to, G: U Wobble or Hoogstein base pairing. Complementary sequences between an oligonucleotide and a target sequence as described herein, include base-pairing of the oligonucleotide or polynucleotide comprising a first nucleotide or nucleoside sequence to an oligonucleotide or polynucleotide comprising a second nucleotide or nucleoside sequence over the entire length of one or both nucleotide or nucleoside sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via an RNase H-mediated pathway. “Substantially complementary” can also refer to a polynucleotide that is substantially complementary to a contiguous portion of the mRNA of interest (e.g., an mRNA encoding a nORF). For example, a polynucleotide is complementary to at least a part of a nORF mRNA if the sequence is substantially complementary to a non-interrupted portion of an mRNA encoding the nORF.

As used herein, the term “region of complementarity” refers to the region on the oligonucleotide that is substantially complementary to all or a portion of a gene, primary transcript, a sequence (e.g., a target sequence, e.g., a nORF nucleotide sequence), or processed mRNA, so as to interfere with expression of the endogenous gene (e.g., nORF). Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the oligonucleotide.

As used herein, “lipid nanoparticle” or “LNP” is a vesicle comprising a lipid layer encapsulating a pharmaceutically active molecule, such as a nucleic acid molecule, e.g., a polynucleotide, e.g., an oligonucleotide. LNP refers to a stable nucleic acid-lipid particle. LNPs typically contain a cationic lipid, a non-cationic lipid, and a lipid that prevents aggregation of the particle (e.g., a PEG-lipid conjugate). LNPs are described in, for example, U.S. Pat. Nos. 6,858,225; 6,815,432; 8,158,601; and 8,058,069, the entire contents of which are hereby incorporated herein by reference.

As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the polynucleotide composition. The lipophilic material isolates the aqueous interior from an aqueous exterior, which typically does not include the oligonucleotide composition, although in some examples, it may. Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids.

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

The term “antisense,” as used herein, refers to a polynucleotide that is sufficiently complementary to all or a portion of a gene, primary transcript, or processed mRNA, so as to interfere with expression of the endogenous gene (e.g., the nORF). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other.

As used herein, the term “region of complementarity” refers to the region on the polynucleotide, e.g., oligonucleotide, that is substantially complementary to all or a portion of a gene, primary transcript, a sequence (e.g., a target sequence, e.g., a nORF sequence), or processed mRNA, so as to interfere with expression of the endogenous gene (e.g., the protein expressed by the nORF). Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the oligonucleotide.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “pharmaceutical composition,” as used herein, represents a composition containing a compound described herein formulated with a pharmaceutically acceptable excipient, and preferably manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for intrathecal injection; for intracerebroventricular injections; for intraparenchymal injection; or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a subject. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

### BRIEF DESCRIPTION OF THE DRAWINGS

FIGS. 1A-1C are graphs showing siRNA knockdown of ENST00000455557.2 (SEQ ID NO: 1) in a head and neck cancer cell line (SCC25). FIG. 1A shows viable cell counts in the presence of a scrambled siRNA (negative control), a positive control (HPRT1), and increasing concentrations of target siRNA. FIG. 1B shows % viable cell counts normalized to time 0 hours in the presence of a scrambled siRNA, a positive control (HPRT1), and increasing concentrations of target siRNA. FIG. 1C shows % knockdown in the presence of a scrambled siRNA (negative control), a positive control (HPRT1), and increasing concentrations of target siRNA.

FIGS. 2A-2C are graphs showing siRNA knockdown of ENST00000383686.3 (SEQ ID NO: 3) in a breast cancer cell line (Hs578T). FIG. 2A shows viable cell counts in the presence of a scrambled siRNA (negative control), a positive control (HPRT1), and increasing concentrations of target siRNA. FIG. 2B shows % viable cell counts normalized to time 0 hours in the presence of a scrambled siRNA, a positive control (HPRT1), and increasing concentrations of target siRNA. FIG. 2C shows % knockdown in the presence of a scrambled siRNA (negative control), a positive control (HPRT1), and increasing concentrations of target siRNA.

FIG. 3 is a table showing SCC25 Platemap and the layout of DsiRNA and ASO probe pools for SCC25 transfection.

FIGS. 4A and 4B are a set of graphs showing knockdown of Target 1 in SCC25 cells by pooled DsiRNAs.

FIGS. 5A and 5B are set of graphs showing knockdown of Target 1 in SCC25 cells by pooled ASOs.

FIGS. 6A and 6B are a set of graphs showing knockdown of Target 1 in Hs578T cells by pooled DsiRNAs or ASOs.

FIGS. 7A and 7B are a set of graphs showing knockdown of Target 3 in Hs578T cells by pooled DsiRNAs.

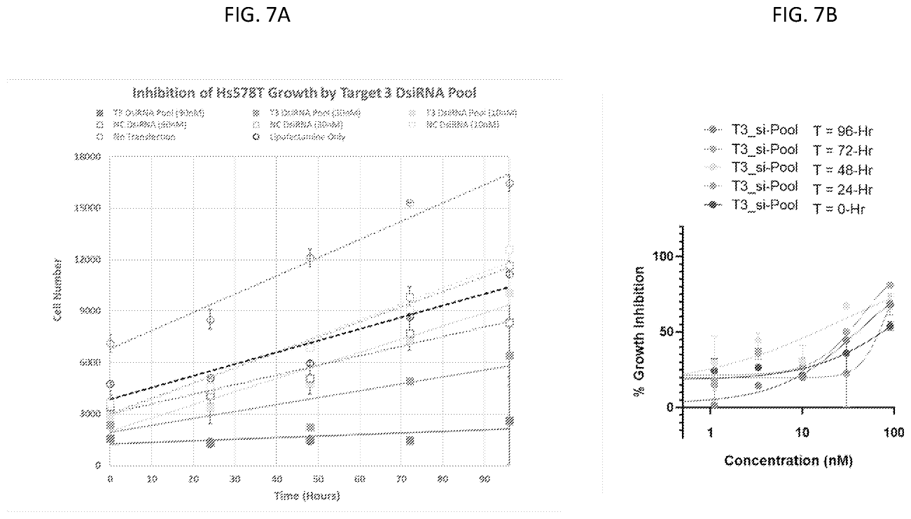

FIG. 8 is a table showing Hs578T Platemap and the layout of DsiRNA and ASO probe pools for Hs578T transfection.

FIGS. 9A and 9B are a set of graphs showing knockdown of Target 3 in Hs578T cells by pooled ASOs.

FIGS. 10A and 10B are a set of graphs showing knockdown of Target 3 in SCC25 cells by pooled DsiRNAs or ASOs.

FIG. 11 is a set of images showing GFP transfection optimization.

FIG. 12 is a set of images showing GFP detection by FLAG-tag immunofluorescence.

FIG. 13 is a set of images showing FLAG-tag detection of NonExomics Targets.

FIG. 14 is a set of images showing target expression using poly-d-lysine coated microplates.

FIG. 15 is a set of images showing Target 2 expressed throughout the cell with no localization.

FIG. 16 is a table showing SCC25 Platemap and the layout of DsiRNA and ASO probe pools for SCC25 transfection.

FIG. 17 is a table showing SCC25 Platemap and the layout of DsiRNA and ASO probe pools for SCC25 transfection.

FIG. 18 is a table showing Hs578T Platemap and the layout of DsiRNA and ASO probe pools for Hs578T transfection.

FIG. 19 is a table showing Hs578T Platemap and the layout of DsiRNA and ASO probe pools for Hs578T transfection.

### DETAILED DESCRIPTION

Described herein are compositions and methods for treating cancer. The present invention is premised, in part, upon the discovery of upregulation of certain novel open reading frames (nORFs) that are distinct from canonical open reading frames (cORF) of genes in cancer cells. By inhibiting expression of the gene that encodes the nORF, the methods described herein can treat the cancer.

### Methods of Treatment

The invention features methods of treating a subject having increased expression of a nORF associated with cancer. In general, the methods feature compositions and methods of reducing expression of the nORFs shown in Table 1 below.

TABLE 1
nORF Sequences

| Name | SEQ ID | Sequence |
|---|---|---|
| ENST00000455557.2 | SEQ ID NO: 1 | ACTGCAAGCTCTGCCTCCCGGGTTCACGCCATTCTCCTGTGTC |
| | | AGCCTCCTGAGTAGCTGGGACTACAGGCGCCCGCCACCATGCC |
| | | CGGCTAATTTTTTTTGTATTTTTAGTAGAGACGGGGTTTCACC |
| | | ATGTTAGCCAGGATAGTTTCAATTTCCGGACCTCGTGATCCGC |
| | | CCGCCTCGGCCTCCCAAAGTGCTGGGATTATAGGCGTGAGCCA |
| | | CCGCGCCCAGCCCCTTTCATTTTTGATATGGAGTTTTGTTCTT |
| | | GTTGCCCAGGCTGGAATGCAATGGCGCGATCTCAGCTCACTGC |
| | | AACCTCTGCCTCCTGGGTTCAAGCGATTCTCCTGGTTCAGCCT |
| | | CCTGAGTAGCTGGGATTACAGGAATGCGCCACCACACCTGGCT |
| | | ACTTTTGTATTTTTAGTAGAGATGGGGTTTCTCCACGTGCATC |
| | | AGGCTAGTCTCACACTCCCGACTTCAGATGATCCACCCGCCTC |
| | | GGCCTCCCAAAGTGCTGGGATTACAGGCGTGAGCCACCGTGCC |
| | | TGGCCAAAGCCTTCTTCACTTTCATTTTGTGGCTGCTGTTGTG |
| | | AAATCTGCAGAACCTCAAGCTGTTTGGACTCCATCTTCTAGGT |
| | | TGAATTAGATTACAGGTAATCTCATGGTGGAGGTGTATTCTGG |
| | | ATCGACTCACTTTTGAAATGTTTATGAAAGAAATTCCTTCACC |
| | | TCGGGACTTCCACACTGGAGCTGGCAATCAGTATCACCTTAAT |
| | | TTACCTATCACAGTGAGGCATCTCTGCTCTGAGGAAAACAAAA |
| | | CAAAACAAAACAAAACAAAAAAGAAGAGCTAATGGTTCTGTTT |
| | | TGTTTTGTTGTTTTAGAGACAGGGTCTTGCTCTGTCGCCCGGG |
| | | CTGGAATGCAGTGACATCATCACAGCTCACTGTAGCCTTGATC |
| | | TCCTGGGCTCAAGCGATCCTTCTGCCTCAGCCTGAGTAGCTGG |
| | | GATGGGACTACAGATGCACGCCACCACACATGGCTAATTTTTT |
| | | TATTTTTTAGAGACGAGGTCCCATGTGAGCCACGGTGCCCCAC |
| | | CTTTTAAAGCAATTACCGTAAGGAAGCAACTGTCTTTAGCAAA |
| | | TAGAAGATCATAAACATCCTTCTGGAGATCTTGAGATGTTGAC |
| | | TGTGGTTTATAGGGCAATGAAAATCTACATACCTGAGTGGGGC |
| | | AGTGAGACATGAGTGCCACCTATGATCCTTCCAGTGAGCAATT |
| | | CTTTCTGGTGCCAGGAGCCCAAAGGAAACTCAGTCAACCAGCT |
| | | GTAGATGGGCTTGTTCCGAGTTTGTTCTAGAAGTCAGAACTGC |
| | | CAGCTCTGAAAGTAGGTCACAAGTGACTGGCCATCCCTGGGCT |
| | | GCTCACAGGAAGTAGCTGCAAAGGTCAGGGAAAGTTTTTAATG |
| | | ACTATACAGGCCAGTGAATGACAGGCTTTGAGGAGCTTCTCTT |
| | | CTTGGCATCTGAAACAGGCACATGGAGGAGCTGCCTTGGAGAC |
| | | AGAATGACAGCTGTGGCAATGTGAGCCTGATTCCTCAGCCCCA |
| | | TAAAATAGATGCCTTTGCAAAAAACCCATTTATTTTTTTTTTT |
| | | TTCTGTTTTTGAGAGGGAGTCTCGCTCTGTCCCCCCAGGCGGA |
| | | GTGCAGTGGCGTGATCTCGGCTCACTGCAACTTCTGCCTCCCG |
| | | GGTTCAAGCGATTCTCCTGCCTCAGCTCCCAAGTAGCTGAGAT |
| | | TACAGGCATCCGCCACCACGCCTGGCCAATTTTGTATTTTAGT |
| | | AGAGACGGGGTTTTGCCACATTGGCCAGGCTGGGAAAAAGCCC |
| | | ATTTCTATACAGAGGAATCATCTTGCAGGACTCAAGGAATGAT |
| | | TAAGAGGAATCTACAGCATTAACTTTAGAAACTCGATGCAATA |
| | | TTCCCAAGAGGAGTTAAGATTGGGATAAGTAATCTCTGAATTA |
| | | ATTTCAACCTTGAGATGTTGTGATCCTATGAAGTCAAAGACAT |
| | | ATTTCTTTTTTCTTTGTTTGAGATGGAGTCTTGCTCTGTCACC |
| | | CAAATTGGAGGGCGGTGATGTGATCTCGGCTTACTACGACCTC |
| | | TGCCTCCTGGGTTCAAGCGATTCTCAGCTTCAGCCTCCTGAGT |
| | | AGCTGAGATTACAGGTGCCCGCCACCATGCCCGGCTAATTTTT |
| | | GTATTTTTAGTAGAGATGAGGTTTCACTGTGTTGGCCAGGCTG |
| | | GTCTTGAATTCTTGGCCCCAGGTGATCTGCCCGCCTGGGCCTC |
| | | CCAAAGTGCTGGGATTACAGGAGTGAGCACCATGTCCGGTCTA |
| | | GATACATATTTCTATTAATACACATTTGCAGGGATTGTTTGTT |
| | | ACTGAAGTTGACGCCTTGTGCTTGAGAATGCTATTTCACTGAC |
| | | ACAATTGTTATGGTATAAATGTGAGAGTATAGTTACAATCTGG |
| | | ACACTATTTTTTGCTTGTCTTATTGGCCCAGCAACCAGCTTGA |
| | | CACTGGGGACTATCAGGCTCCAAATAATAACCAATGTCTCACT |
| | | CCAAACAGACAGGATACTACGGAGCCAGGGTCAGCAAACATTT |
| | | TCTGTAAAGGGCCAGATAGTAAATATTTTGGGCTTTGTGGGCC |
| | | CTATGGTCTCTGTCACAACGATTCAACTCTGCTGTTGTAACAA |
| | | AAAAGCAGTCAGACAATATGTAAATGAATGGGCATGGCTGTGT |
| | | TCCAGTAAAACCACAAAAACAGGCAGCTAGCTTTGGTGTGGGG |
| | | CCCTAAGAGCTTGGACTTTGAAGCCAGGTTCAAATCCTGGCTT |
| | | TCTCACCTTCTAGCTCCGTTACCTTAAAGAACTTCATTATCTT |
| | | CCTTGTATTTTAGTCTTTTCATCTCTAAATTAGTGATAGTACT |
| | | TAACAGTGTTGTTATAAAGATTAAACGAGTTAAGTTTGTAAAG |
| | | TACTTAAACAGTGCCTACCAGGTAGTAAGTGCTATAGCAGAGT |
| | | GTGTTAGATAATGAAACATCCTGTGCTGTGGATATATTATTCC |
| | | CATTACGATTTGGATTCAAATAAAGAAATACATGACTTAGAAG |
| | | AA |
| ENST00000455557.2 | SEQ ID NO: 2 | MLARIVSISGPRDPPASASQSAGIIGVSHRAQPLSFLIWSFV |
| Protein Sequence | | LVAQAGMQWRDLSSLQPLPPGFKRFSWFSLLSSWDYRNAPPH |
| | | LATFVFLVEMGFLHVHQASLTLPTSDDPPASASQSAGITGVS |
| | | HRAWPKPSSLSFCGCCCEICRISSCLDSIF |
| ENST00000383686.3 | SEQ ID NO: 3 | GAGAGATTTCATCTACGGGAAAAAGACCCTCTGAAAAGAAGTG |
| | | TGAAATCATCAAAAGAGCCATTGGAGGGGACACCAGAGAGTCG |
| | | TGTGGCTGAGGTTTCACATGATCCCGCCATGCTTCCAGTCCAG |
| | | AGGAGAAAGCTAGACCCACTCCTCAAAAAGTATAGGCATCATA |
| | | AGAAGGCCACAAGGACAAAAAGAAGGAGGAAGGAGAAAATGGA |
| | | GGCCCAGTGCTCCCCTGTTCCTCCAACACCTTCAACACCACCA |
| | | CAAAGTGAAGAAGATGAGGCTGTAGACAAAAAGCCAACTCTAC |
| | | TCAGTGCCCAGGAAGATACTCCTGACCTTCTCCATGAGGACAG |
| | | ATTACAGTATCTGCAGGAAGAGGGTTCCGGTGTGATGCATCAG |
| | | GAATGTCAGATCCAGTCCTGTGAGCTCTCGGTGGCTCAGAAGC |
| | | CCAGGCCCTCTTCTCCTGCAGTGACATCCTTGGCATCACCACC |
| | | ACTCTGCTTTGGCAGTTTCCTAAGCTGTGTCTGCCAGACCTTC |
| | | TCAAGGTCTAGGAAGCAGAAGCCTCCCAGAAGAAAGGGTAACA |
| | | ACCAGGCTGAGGCAGGAGGTGATGCTGAGGTCCTGAGACCTGG |
| | | CCCGGCAAAACCAGAGTTGAGCCTCAGCAAAACGTGTAGCCAC |
| | | TTGAAATCGATAGAGCTTTCCTTTGTGTTTAGTTTTATAGTTC |
| | | TCTCTGTATGTCATTGTTCCTCTTAGTTGAGTTTTTGCCTGTC |
| | | AAAGAGATAGAGAGCTGCTTTTATGCTGAGATCCTCAAGCCAA |
| | | AACCCACAGTAAACGCAAAGTAAAACTTT |
| ENST00000383686.3 | SEQ ID NO: 4 | MLPVQRRKLDPLLKKYRHHKKATRTKRRRKEKMEAQCSPVPP |
| Protein Sequence | | TPSTPPQSEEDEAVDKKPTLLSAQEDTPDLLHEDRLQYLQEE |
| | | GSGVMHQECQIQSCELSVAQKPRPSSPAVTSLASPPLCFGSF |
| | | LSCVCQTFSRSRKQKPPRRKGNNQAEAGGDAEVLRPGPAKPE |
| | | LSLSKTCSHLKSIELSFVFSFIVLSVCHCSS |

The invention features a method of treating cancer in a subject by administering a polynucleotide that reduces expression of the nORF of SEQ ID NO: 1 or its protein product of SEQ ID NO: 2. In some embodiments, the invention features a method of treating cancer by administering a polynucleotide that targets any one or more of nucleotides 180-597 of SEQ ID NO: 1. In some embodiments, the polynucleotide targets one or more of nucleotides 130-180, 180-230, 230-280, 280-330, 330-380, 380-430, 430-480, 480-530, 530-580, or 580-597 of SEQ ID NO: 1. For example, the polynucleotide may target one or more of nucleotides 130-150, 150-170, 170-190, 190-210, 210-230, 230-250, 250-270, 270-290, 290-310, 310-330, 330-350, 350-370, 370-390, 390-410, 410-430, 430-450, 450-470, 470-490, 490-510, 510-530, 530-550, 550-570, 570-590, or 590-597 of SEQ ID NO: 1. In some embodiments, the polynucleotide has at least 80% (e.g., at least 85%, 90%, 95%, 97%, 99%, or 100%) sequence identity to any one of SEQ ID NOs: 5-13. In some embodiments, the polynucleotide includes the sequence of any one of SEQ ID NOs: 5-13.

The cancer may be, for example, head and neck cancer, bladder urothelial carcinoma, breast invasive carcinoma, cervical cancer, endocervical cancer, colon adenocarcinoma, esophageal carcinoma, kidney clear cell carcinoma, kidney papillary cell carcinoma, lung adenocarcinoma, lung squamous cell carcinoma, ovarian serous cystadenocarcinoma, pancreatic adenocarcinoma, rectum adenocarcinoma, skin cutaneous melanoma, stomach adenocarcinoma, uterine carcinosarcoma, or uterine corpus endometrioid carcinoma. In some embodiments, the cancer is head and neck cancer.

The invention also features a method of treating cancer in a subject by administering a polynucleotide that reduces expression of the nORF of SEQ ID NO: 3 or its protein product of SEQ ID NO: 4. In some embodiments, the invention features a method of treating cancer by administering a polynucleotide that targets any one or more of nucleotides 115-711 of SEQ ID NO: 3. In some embodiments, the polynucleotide targets one or more of nucleotides 115-165, 165-215, 215-265, 265-315, 315-365, 365-415, 415-465, 465-515, 515-565, 565-615, 615-665, or 665-711 of SEQ ID NO: 3. For example, the polynucleotide may target one or more of nucleotides 115-135, 135-155, 155-175, 175-195, 195-215, 215-235, 235-255, 255-275, 275-295, 295-315, 315-335, 335-355, 355-375, 375-395, 395-415, 415-435, 435-455, 455-475, 475-495, 495-515, 515-535, 535-555, 555-575, 575-595, 595-615, 615-635, 635-655, 655-675, 675-695, or 695-711 of SEQ ID NO: 3. In some embodiments, the polynucleotide has at least 80% (e.g., at least 85%, 90%, 95%, 97%, 99%, or 100%) sequence identity to any one of SEQ ID NOs: 14-22. In some embodiments, the polynucleotide includes the sequence of any one of SEQ ID NOs: 14-22.

The cancer may be, for example, brain lower grade glioma, breast invasive carcinoma, diffuse large B cell lymphoma, esophageal carcinoma, kidney clear cell carcinoma, kidney papillary cell carcinoma, liver hepatocellular carcinoma, lung adenocarcinoma, lung squamous cell carcinoma, ovarian serous cystadenocarcinoma, pancreatic adenocarcinoma, prostate adenocarcinoma, skin cutaneous melanoma, stomach adenocarcinoma, testicular germ cell tumor, thymoma, thyroid carcinoma, uterine carcinosarcoma, or uterine corpus endometrioid carcinoma. In some embodiments, the cancer is breast invasive carcinoma.

These nORFS may exhibit an increase (e.g., by at least 5%, 10%, 15%, 20%, 25%, 30%, 40%, 50%, 60%, 70%, 80%, 90%, 100%, 200%, 300%, 400%, 500%, or more) in expression, e.g., as compared to the nORF in normal (e.g., noncancerous) tissue or compared to a normal subject without cancer. The subject may be first determined to have the dysregulated nORF and then may subsequently be treated for the cancer. The subject may have previously been determined to have the dysregulated nORF and is then treated for the cancer. The polynucleotide that targets the dysregulated nORF may decrease (e.g., by at least 5%, 10%, 15%, 20%, 25%, 30%, 40%, 50%, 60%, 70%, 80%, 90%, 95%, 97%, or 99%) expression of the upregulated nORF.

The methods described herein feature methods of treating cancer. The present invention contemplates treatment of a cancer in which a nORF (e.g., of SEQ ID NOs: 1-4) exhibits increased expression, e.g., relative to a noncancerous cell.

The method may reduce the size (e.g., by 5%, 10%, 15%, 20%, 25%, 30%, 40%, 50%, 60%, 70%, 80%, 90%, 95%, 97%, or 99%) of a tumor in the or breast. The method may decrease or slow (e.g., by at least 5%, 10%, 15%, 20%, 25%, 30%, 40%, 50%, 60%, 70%, 80%, 90%, 95%, 97%, or 99%) the progression of cancer. The method may decrease (e.g., by at least 5%, 10%, 15%, 20%, 25%, 30%, 40%, 50%, 60%, 70%, 80%, 90%, 95%, 97%, or 99%) the risk of developing cancer. The method may decrease (e.g., by at least 5%, 10%, 15%, 20%, 25%, 30%, 40%, 50%, 60%, 70%, 80%, 90%, 95%, 97%, or 99%) the risk of developing a relapse of cancer.

### Polynucleotide Inhibitors

The methods of treatment described herein include providing a polynucleotide that targets the upregulated nORF (e.g., SEQ ID NO: 1 or 3, e.g., one or more of nucleotides 130-597 of SEQ ID NO: 1 or one or more of nucleotides 115-711 of SEQ ID NO: 3). The inhibitor may reduce (e.g., by at least 5%, 10%, 15%, 20%, 25%, 30%, 40%, 50%, 60%, 70%, 80%, 90%, 95%, 97%, or 99%) an amount or activity of the dysregulated nORF, such as to prevent the deleterious effect of the dysregulated nORF. Suitable polynucleotides that can reduce an amount or activity of the dysregulated nORF include RNA. For example, an RNA for reducing an activity or amount of the upregulated nORF may be, for example, a miRNA, an antisense polynucleotide, an shRNA, or an siRNA. The miRNA, antisense polynucleotide, shRNA, or siRNA may target a region of RNA (e.g., dysregulated nORF gene) to reduce expression of the dysregulated nORF. The polynucleotide may be provided directly or may be provided by a vector (e.g., a viral vector) encoding the polynucleotide. The polynucleotide inhibitor may be formulated, e.g., in a pharmaceutical composition containing a pharmaceutically acceptable carrier. The composition can be administered by any suitable method known in the art to the skilled artisan. The composition (e.g., a vector, e.g., a viral vector) may be formulated in a virus or a virus-like particle.

The polynucleotides described herein may target any one or more of nucleotides 130-597 of SEQ ID NO: 1. In some embodiments, the polynucleotide targets one or more of nucleotides 130-180, 180-230, 230-280, 280-330, 330-380, 380-430, 430-480, 480-530, 530-580, or 580-597 of SEQ ID NO: 1. For example, the polynucleotide may target one or more of nucleotides 130-150, 150-170, 170-190, 190-210, 210-230, 230-250, 250-270, 270-290, 290-310, 310-330, 330-350, 350-370, 370-390, 390-410, 410-430, 430-450, 450-470, 470-490, 490-510, 510-530, 530-550, 550-570, 570-590, or 590-597 of SEQ ID NO: 1. In some embodiments, the polynucleotide has at least 80% (e.g., at least 85%, 90%, 95%, 97%, 99%, or 100%) sequence identity to any one of SEQ ID NOs: 5-13. In some embodiments, the polynucleotide includes the sequence of any one of SEQ ID NOs: 5-13.

In some embodiments, the polynucleotide has a nucleobase sequence including a portion of at least 10 (e.g., at least 11, 12, 13, 14, 15, 16, 17, 18, 19, 20, 25, 30, 35, 40, 45, 50, or more) contiguous nucleobases having at least 80% (e.g., at least 85%, 90%, 95%, 97%, 99%, or 100%) complementarity to an equal length portion of nucleotides 130-597 of SEQ ID NO: 1.

The polynucleotides described herein may target any one or more of nucleotides 115-711 of SEQ ID NO: 3. In some embodiments, the polynucleotide targets one or more of nucleotides 115-165, 165-215, 215-265, 265-315, 315-365, 365-415, 415-465, 465-515, 515-565, 565-615, 615-665, or 665-711 of SEQ ID NO: 3. For example, the polynucleotide may target one or more of nucleotides 115-135, 135-155, 155-175, 175-195, 195-215, 215-235, 235-255, 255-275, 275-295, 295-315, 315-335, 335-355, 355-375, 375-395, 395-415, 415-435, 435-455, 455-475, 475-495, 495-515, 515-535, 535-555, 555-575, 575-595, 595-615, 615-635, 635-655, 655-675, 675-695, or 695-711 of SEQ ID NO: 3. In some embodiments, the polynucleotide has at least 80% (e.g., at least 85%, 90%, 95%, 97%, 99%, or 100%) sequence identity to any one of SEQ ID NOs: 14-22. In some embodiments, the polynucleotide includes the sequence of any one of SEQ ID NOs: 14-22.

In some embodiments, the polynucleotide has a nucleobase sequence including a portion of at least 10 (e.g., at least 11, 12, 13, 14, 15, 16, 17, 18, 19, 20, 25, 30, 35, 40, 45, 50, or more) contiguous nucleobases having at least 80% (e.g., at least 85%, 90%, 95%, 97%, 99%, or 100%) complementarity to an equal length portion of nucleotides 115-711 of SEQ ID NO: 3.

Using the compositions and methods described herein, a patient with a cancer may be administered a polynucleotide, a composition containing the same, or a vector encoding the same, so as to reduce or suppress the expression of a dysregulated (e.g., upregulated) nORF. Exemplary interfering RNA molecules that may be used in conjunction with the compositions and methods described herein are siRNA molecules, miRNA molecules, shRNA molecules, and antisense polynucleotide molecules, among others. In the case of siRNA molecules, the siRNA may be single stranded or double stranded. miRNA molecules, in contrast, are single-stranded molecules that form a hairpin, thereby adopting a hydrogen-bonded structure reminiscent of a nucleic acid duplex. In either case, the interfering RNA may contain an antisense or “guide” strand that anneals (e.g., by way of complementarity) to the repeat-expanded mutant RNA target. The interfering RNA may also contain a “passenger” strand that is complementary to the guide strand and, thus, may have the same nucleic acid sequence as the RNA target.

siRNA is a class of short (e.g., 20-25 nt) double-stranded non-coding RNA that operates within the RNA interference pathway. siRNA may interfere with expression of the dysregulated nORF gene with complementary nucleotide sequences by degrading mRNA (via the Dicer and RISC pathways) after transcription, thereby preventing translation. miRNA is another short (e.g., about 22 nucleotides) non-coding RNA molecule that functions in RNA silencing and post-transcriptional regulation of gene expression. miRNAs function via base-pairing with complementary sequences within mRNA molecules, thereby leading to cleavage of the mRNA strand into two pieces and destabilization of the mRNA through shortening of its poly (A) tail. shRNA is an artificial RNA molecule with a tight hairpin turn that can be used to silence target gene expression via RNA interference. Antisense polynucleotides are also short single stranded molecules that hybridize to a target RNA and prevent translation by occluding the translation machinery, thereby reducing expression of the target (e.g., the dysregulated nORF).

siRNA

The siRNA molecules of the disclosure may be in the form of a single-stranded (ss) or double-stranded (ds) RNA structure. In some embodiments, the siRNA molecules may be di-branched, tri-branched, or tetra-branched molecules. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest siRNAs consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNA interference (RNAi) activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

siRNAs described herein may employ modifications to the nucleobase, phosphate backbone, ribose core, 5′- and 3′-ends, and branching, wherein multiple strands of siRNA may be covalently linked.

It is within the scope of the disclosure that any length, known and previously unknown in the art, may be employed for the current invention. As described herein, potential lengths for an antisense strand of the polynucleotides of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand (e.g., of an siRNA) of the polynucleotides of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### Antisense Polynucleotides

Agents described herein that reduce the level and/or activity of the nORF in a cell may be, for example, an antisense polynucleotide, e.g., an antisense oligonucleotide. In some embodiments, the polynucleotide is a single-stranded oligonucleotide, e.g., that acts by way of an RNase H-mediated pathway. Oligonucleotides include DNA and DNA/RNA chimeric molecules, typically about 10 to 30 nucleotides in length, which recognize polynucleotide target sequences or sequence portions through hydrogen bonding interactions with the nucleotide bases of the target sequence (e.g., the nORF). An oligonucleotide molecule can decrease the expression level (e.g., protein level or mRNA level) of the nORF. For example, an oligonucleotide includes oligonucleotides that targets the coding region of the nORF. In some embodiments, the oligonucleotide molecule recruits an RNase H enzyme, leading to target mRNA degradation.

The oligonucleotide includes a region of complementarity (e.g., a contiguous nucleobase region), which is complementary to at least a part of an mRNA formed in the expression of a nORF gene. The region of complementarity may be about 30 nucleotides or less in length (e.g., about 30, 29, 28, 27, 26, 25, 24, 23, 22, 21, 20, 19, or 18 nucleotides or less in length). Upon contact with a cell expressing the nORF gene, the oligonucleotide may inhibit the expression of the nORF gene by at least about 10% as assayed by, for example, a PCR or branched DNA (bDNA)-based method, or by a protein-based method, such as by immunofluorescence analysis, using, for example, Western Blotting or flow cytometric techniques.

The antisense oligonucleotide may be between 10 and 80 nucleotides, 10 and 50 nucleotides, 12 and 40 nucleotides, 15 and 40 nucleotides, 20 and 30 nucleotides, 16 and 30 nucleotides, 18 and 22 nucleotides, 15 and 25 nucleotides, 18 and 25 nucleotides, 18 and 22 nucleotides, or 18 and nucleotides. For example, the oligonucleotide may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. The region of complementarity to the target sequence may be between 10 and 30 linked nucleosides in length, e.g., between 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 linked nucleosides in length. Ranges and lengths intermediate to the above recited ranges and lengths are also contemplated to be part of the invention.

In one aspect, an oligonucleotide includes a region of at least 10 (e.g., at least 11, 12, 13, 14, 15, 16, 17, 18, 19, 20, 25, 30, 35, 40, 45, 50, or more) contiguous nucleobases having at least 80% (e.g., at least 85%, at least 90%, at least 95%, or at least 99%) complementary to at least 10 (e.g., at least 11, 12, 13, 14, 15, 16, 17, 18, 19, 20, 25, 30, 35, 40, 45, 50, or more) contiguous nucleotides of a nORF gene (e.g., SEQ ID NO: 1 or 3, e.g., nucleotides 130-597 of SEQ ID NO: 1 or nucleotides 115-711 of SEQ ID NO: 3). In some embodiments, the oligonucleotide includes a sequence complementary to at least 17 contiguous nucleotides, 19-23 contiguous nucleotides, 19 contiguous nucleotides, or 20 contiguous nucleotides of a nORF gene.

In some embodiments the oligonucleotide of the invention, or contiguous nucleotide region thereof, has a gapmer design or structure also referred herein merely as “gapmer.” In a gapmer structure the oligonucleotide includes at least three distinct structural regions a 5′-wing, a gap and a 3′-wing, in ‘5->3’ orientation. In this design, the 5′ and 3′ wing regions (also termed flanking regions) include at least one alternative nucleoside which is adjacent to a gap region and may in some embodiments include a contiguous stretch of 2-7 alternative nucleosides, or a contiguous stretch of alternative and DNA nucleosides (mixed wings including both alternative and DNA nucleosides). The length of the 5′-wing region may be at least two nucleosides in length (e.g., at least at least 2, at least 3, at least 4, at least 5, or more nucleosides in length). The length of the 3′-wing region may be at least two nucleosides in length (e.g., at least 2, at least 3, at least at least 4, at least 5, or more nucleosides in length). The 5′ and 3′ wing regions may be symmetrical or asymmetrical with respect to the number of nucleosides they include. In some embodiments, the gap region includes about 10 nucleosides flanked by a 5′ and a 3′ wing region each including about 5 nucleosides, also referred to as a 5-10-5 gapmer.

Consequently, the nucleosides of the 5′ wing region and the 3′ wing region which are adjacent to the gap region are alternative nucleosides, such as 2′ alternative nucleosides. The gap region includes a contiguous stretch of nucleotides which are capable of recruiting RNase H, when the oligonucleotide is in duplex with the nORF target nucleic acid. In some embodiments, the gap region includes a contiguous stretch of 5-16 DNA nucleosides. In other embodiments, the gap region includes a region of at least 10 (e.g., at least 11, 12, 13, 14, 15, 16, 17, 18, 19, 20, 25, 30, 35, 40, 45, 50, or more) contiguous nucleobases having at least 80% (e.g., at least 85%, at least 90%, at least 95%, or at least 99%) complementarity to a nORF gene. In some embodiments, the gapmer includes a region complementary to at least 17 contiguous nucleotides, 19-23 contiguous nucleotides, or 19 contiguous nucleotides of a nORF gene. The gapmer is complementary to the nORF target nucleic acid and may therefore be the contiguous nucleoside region of the oligonucleotide.

The 5′ and 3′ wing regions, flanking the 5′ and 3′ ends of the gap region, may include one or more affinity enhancing alternative nucleosides. In some embodiments, the 5′ and/or 3′ wing includes at least one 2′-O-methoxyethyl (MOE) nucleoside, preferably at least two MOE nucleosides. In some embodiments, the 5′ wing includes at least one MOE nucleoside. In some embodiments both the 5′ and 3′ wing regions include an MOE nucleoside. In some embodiments all the nucleosides in the wing regions are MOE nucleosides. In other embodiments, the wing regions may include both MOE nucleosides and other nucleosides (mixed wings), such as DNA nucleosides and/or non-MOE alternative nucleosides, such as bicyclic nucleosides (BNAs) (e.g., LNA nucleosides or cET nucleosides), or other 2′ substituted nucleosides. In this case the gap is defined as a contiguous sequence of at least 5 RNase H recruiting nucleosides (such as 5-16 DNA nucleosides) flanked at the 5′ and 3′ end by an affinity enhancing alternative nucleoside, such as an MOE nucleoside.

In other embodiments, the 5′ and/or 3′ wing includes at least one BNA (e.g., at least one LNA nucleoside or cET nucleoside), preferably at least 2 bicyclic nucleosides. In some embodiments, the 5′ wing includes at least one BNA. In some embodiments both the 5′ and 3′ wing regions include a BNA. In some embodiments all the nucleosides in the wing regions are BNAs. In other embodiments, the wing regions may include both BNAs and other nucleosides (mixed wings), such as DNA nucleosides and/or non-BNA alternative nucleosides, such as 2′ substituted nucleosides. In this case the gap is defined as a contiguous sequence of at least five RNase H recruiting nucleosides (such as 5-16 DNA nucleosides) flanked at the 5′ and 3′ end by an affinity enhancing alternative nucleoside, such as a BNA, such as an LNA, such as beta-D-oxy-LNA.

The 5′ flank or 5′ wing attached to the 5′ end of the gap region includes, contains, or consists of at least one alternative sugar moiety (e.g., at least three, at least four, at least five, at least six, at least seven, or more alternative sugar moieties). In some embodiments the wing region includes or consists of from 1 to 7 alternative nucleobases, such as from two to six alternative nucleobases, from two to five alternative nucleobases, from two to four alternative nucleobases, or from one to three alternative nucleobases (e.g., one, two, three or four alternative nucleobases). In some embodiments, the wing region includes or consists of at least one alternative internucleoside linkage (e.g., at least three, at least four, at least five, at least six, at least seven, or more alternative internucleoside linkages).

The 3′ flank or 3′ wing attached to the 3′ end of the gap region includes, contains, or consists of at least one alternative sugar moiety (e.g., at least three, at least four, at least five, at least six, at least seven, or more alternative sugar moieties). In some embodiments the wing region includes or consists of from one to seven alternative nucleobases, such as from two to six alternative nucleobases, from two to five alternative nucleobases, from two to four alternative nucleobases, or from one to three alternative nucleobases (e.g., two, three, or four alternative nucleobases). In some embodiments, the wing region includes or consists of at least one alternative internucleoside linkage (e.g., at least three, at least four, at least five, at least six, at least seven, or more alternative internucleoside linkages).

In an embodiment, one or more or all of the alternative sugar moieties in the wing regions are 2′ alternative sugar moieties.

In a further embodiment, one or more of the 2′ alternative sugar moieties in the wing regions are selected from 2′-O-alkyl-sugar moieties, 2′-O-methyl-sugar moieties, 2′-amino-sugar moieties, 2′-fluoro-sugar moieties, 2′-alkoxy-sugar moieties, MOE sugar moieties, LNA sugar moieties, arabino nucleic acid (ANA) sugar moieties, and 2′-fluoro-ANA sugar moieties.

In one embodiment of the invention all the alternative nucleosides in the wing regions are bicyclic nucleosides. In a further embodiment the bicyclic nucleosides in the wing regions are independently selected from the group consisting of oxy-LNA, thio-LNA, amino-LNA, cET, and/or ENA, in either the beta-D or alpha-L configurations or combinations thereof.

In some embodiments, the one or more alternative internucleoside linkages in the wing regions are phosphorothioate internucleoside linkages. In some embodiments, the phosphorothioate linkages are stereochemically pure phosphorothioate linkages. In some embodiments the phosphorothioate linkages are Sp phosphorothioate linkages. In other embodiments, the phosphorothioate linkages are Rp phosphorothioate linkages. In some embodiments, the alternative internucleoside linkages are 2′-alkoxy internucleoside linkages. In other embodiments, the alternative internucleoside linkages are alkyl phosphate internucleoside linkages.

The gap region may include, contain, or consist of at least 5-16 consecutive DNA nucleosides capable of recruiting RNase H. In some embodiments, all of the nucleosides of the gap region are DNA units. In further embodiments the gap region may consist of a mixture of DNA and other nucleosides capable of mediating RNase H cleavage. In some embodiments, at least 50% of the nucleosides of the gap region are DNA, such as at least 60%, at least 70% or at least 80%, or at least 90% DNA.

The oligonucleotide of the invention includes a contiguous region which is complementary to the target nucleic acid. In some embodiments, the oligonucleotide may further include additional linked nucleosides positioned 5′ and/or 3′ to either the 5′ and 3′ wing regions. These additional linked nucleosides can be attached to the 5′ end of the 5′ wing region or the 3′ end of the 3′ wing region, respectively. The additional nucleosides may, in some embodiments, form part of the contiguous sequence, which is complementary to the target nucleic acid, or in other embodiments, may be non-complementary to the target nucleic acid.

The inclusion of the additional nucleosides at either, or both of the 5′ and 3′ wing regions may independently include one, two, three, four, or five additional nucleotides, which may be complementary or non-complementary to the target nucleic acid. In this respect the oligonucleotide of the invention, may in some embodiments include a contiguous sequence capable of modulating the target which is flanked at the 5′ and/or 3′ end by additional nucleotides. Such additional nucleosides may serve as a nuclease susceptible biocleavable linker and may therefore be used to attach a functional group such as a conjugate moiety to the oligonucleotide of the invention. In some embodiments the additional 5′ and/or 3′ end nucleosides are linked with phosphodiester linkages and may be DNA or RNA. In another embodiment, the additional 5′ and/or 3′ end nucleosides are alternative nucleosides which may for example be included to enhance nuclease stability or for ease of synthesis.

micro-RNA (miRNA)

miRNAs of the disclosure are single stranded (ss) nucleic acid molecules made of DNA, RNA, or both DNA and RNA (e.g., a chimeric) that are complementary to a target gene of interest and prevent translation of the target's mRNA into a protein. Once a miRNA molecule enters a cell, it is incorporated into an RNA-induced silencing complex (RISC). Upon miRNA hybridization to a target mRNA, the RISC complex will cleave the target mRNA, thereby inactivating the target mRNA, resulting in reduced mRNA and protein levels of the target.

In some embodiments, miRNAs of the disclosure may include a nucleotide sequence of 6 to 30 nucleotides in length (e.g., 5, 6, 7, 8, 9, 10, 11, 12, 13, 14, 15, 16, 17, 18, 19, 20, 21, 22, 23, 24, 25, 26, 27, 28, 29, 30, or 31 nucleotides in length).

The nucleotide sequence of the miRNA may contain sufficient complementary to a portion of a target gene of interest (e.g., e.g., SEQ ID NO: 1 or 3, e.g., nucleotides 130-597 of SEQ ID NO: 1 or nucleotides 115-711 of SEQ ID NO: 3) such that the miRNA can hybridize with the target gene of interest. In some embodiments, the miRNA is at least 70%, at least 75%, at least 80%, at least 85%, at least 90%, at least 95%, at least 96%, at least 97%, at least 98%, or at least 99% complementary to the target gene of interest (e. e.g., e.g., SEQ ID NO: 1 or 3, e.g., nucleotides 130-597 of SEQ ID NO: 1 or nucleotides 115-711 of SEQ ID NO: 3) or a portion thereof. In some embodiments, the miRNA is 100% complementary to the target gene of interest (e.g., e.g., SEQ ID NO: 1 or 3, e.g., nucleotides 130-597 of SEQ ID NO: 1 or nucleotides 115-711 of SEQ ID NO: 3) or a portion thereof.

In some embodiments, the nucleotide sequence of the miRNA may contain sufficient complementary to an exon sequence of a target gene of interest (e.g., an exon of the nORF). In some embodiments, the nucleotide sequence of the miRNA may contain sufficient complementary to an intron sequence of a target gene of interest (e.g., an intron of the nORF). In some embodiments, the miRNA of the disclosure may contain sufficient complementarity to a pre-mRNA transcript or an mRNA transcript encoding SEQ ID NO: 1 or 3, e.g., nucleotides 130-597 of SEQ ID NO: 1 or nucleotides 115-711 of SEQ ID NO: 3.

Different miRNAs can be combined for decreasing the protein expression of a target gene of interest. A combination of two or more miRNAs may be used in a method of the invention, such as two different miRNAs, three different miRNAs, four different miRNAs, or five different miRNAs targeting the same gene of interest (e.g., AC9, or variants thereof)

shRNA

shRNAs of the disclosure are ss or ds nucleic acid molecules made of DNA, RNA, or both DNA and RNA (e.g., a chimeric) that are complementary to a target nORF of interest and prevent translation of the target's mRNA into a protein. Once a shRNA molecule enters a cell, it is incorporated into an RNA-induced silencing complex (RISC). Upon shRNA hybridization to a target mRNA, the RISC complex will cleave the target mRNA, thereby inactivating the target mRNA, resulting in reduced mRNA and protein levels of the target.

In some embodiments, shRNAs of the disclosure may include a nucleotide sequence of 60 to 100 nucleotides in length (e.g., 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length). shRNAs of the disclosure contain a variable hairpin loop structure and a stem sequence. In some embodiments the stem sequence may be 10 to 50 nucleotides in length (e.g., 18, 19, 20, 21, 22, 23, 24, 25, 26, 27, 28, 29, or 30 nucleotides in length). In some embodiments, the hairpin size is between 4 to 50 nucleotides in length, although the loop size may be larger without significantly affecting silencing activity. shRNA molecules of the disclosure may contain mismatches, for example G-U mismatches between two strands of the shRNA stem without decreasing potency. In some embodiments, shRNAs are designed to include one or several G-U pairings in the hairpin stem to stabilize hairpins during propagation in bacteria, for example.

The nucleotide sequence of the shRNA may contain sufficient complementary to a portion of a target gene of interest (e.g., e.g., SEQ ID NO: 1 or 3, e.g., nucleotides 130-597 of SEQ ID NO: 1 or nucleotides 115-711 of SEQ ID NO: 3) such that the shRNA can hybridize with the target gene of interest. In some embodiments, the shRNA is at least 70%, at least 75%, at least 80%, at least 85%, at least 90%, at least 95%, at least 96%, at least 97%, at least 98%, or at least 99% complementary to the target gene of interest (e.g., e.g., SEQ ID NO: 1 or 3, e.g., nucleotides 130-597 of SEQ ID NO: 1 or nucleotides 115-711 of SEQ ID NO: 3) or a portion thereof. In some embodiments, the shRNA is 100% complementary to the target gene of interest (e.g., e.g., SEQ ID NO: 1 or 3, e.g., nucleotides 130-597 of SEQ ID NO: 1 or nucleotides 115-711 of SEQ ID NO: 3) or a portion thereof.

In some embodiments, the nucleotide sequence of the shRNA may contain sufficient complementary to an exon sequence of a target gene of interest (e.g., an exon of the nORF). In some embodiments, the nucleotide sequence of the shRNA may contain sufficient complementary to an intron sequence of a target gene of interest (e.g., an intron of the nORF). In some embodiments, the shRNA of the disclosure may contain sufficient complementarity to a pre-mRNA transcript or an mRNA transcript encoding the nORF.

Different shRNAs can be combined for decreasing the protein expression of a target gene of interest (e.g., AC9). A combination of two or more shRNAs may be used in a method of the invention, such as two different shRNAs, three different shRNAs, four different shRNAs, or five different shRNAs targeting the same nORF.

### 2′ Sugar Modifications

The polynucleotides described herein may include at least one (e.g., at least 1, 2, 3, 4, 5, 6, 7, 8, 9, 10, 11, or more) nucleosides having 2′ sugar modifications. Possible 2′-modifications include all possible orientations of OH; F; O-, S-, or N-alkyl; O-, S-, or N-alkenyl; O—, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl (2′-O-Me) modification. Some embodiments use O [(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the polynucleotide, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

Polynucleotides may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. Polynucleotides of the present disclosure may also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-1,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### Internucleoside Linkage Modifications

Another variable in the design of the present disclosure is the internucleoside linkage making up the phosphate backbone of the polynucleotide. Although the natural RNA phosphate backbone may be employed here, derivatives thereof may be used which enhance desirable characteristics of the polynucleotide. Although not limiting, of particular importance in the present disclosure is protecting parts, or the whole, of the polynucleotides from hydrolysis. One example of a modification that decreases the rate of hydrolysis is phosphorothioates. Any portion or the whole of the backbone may contain phosphate substitutions (e.g., phosphorothioates, phosphodiesters, etc.). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential polynucleotides useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′,5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

### Viral Vectors for Expression

Viral genomes provide a rich source of vectors that can be used for the efficient delivery of exogenous genes into a mammalian cell. The gene to be delivered may include an inhibitor that targets a dysregulated nORF, such as an RNA (e.g., a miRNA, an antisense polynucleotide, an shRNA, or an siRNA). Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., an adeno-associated viral (AAV) vector), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### Retroviral Vectors

The delivery vector used in the methods described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene encoding the polypeptide or RNA. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the agent of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther.; 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

The viral vectors (e.g., retroviral vectors, e.g., lentiviral vectors) may include a promoter operably coupled to the transgene encoding the polypeptide or the polynucleotide encoding the RNA to control expression. The promoter may be a ubiquitous promoter. Alternatively, the promoter may be a tissue specific promoter, such as a myeloid cell-specific or hepatocyte-specific promoter. Suitable promoters that may be used with the compositions described herein include CD11b promoter, sp146/p47 promoter, CD68 promoter, sp146/gp9 promoter, elongation factor 1 α (EF1α) promoter, EF1α short form (EFS) promoter, phosphoglycerate kinase (PGK) promoter, α-globin promoter, and β-globin promoter. Other promoters that may be used include, e.g., DC172 promoter, human serum albumin promoter, alpha1 antitrypsin promoter, thyroxine binding globulin promoter. The DC172 promoter is described in Jacob, et al. Gene Ther. 15:594-603, 2008, hereby incorporated by reference in its entirety.

The viral vectors (e.g., retroviral vectors, e.g., lentiviral vectors) may include an enhancer operably coupled to the transgene encoding the polypeptide or the polynucleotide encoding the RNA to control expression. The enhancer may include a β-globin locus control region (BLCR).

Methods of Measuring nORF Gene Expression

Preferably, the compositions and methods of the disclosure are used to facilitate expression of a nORF at physiologically normal levels in a patient (e.g., a human patient), e.g., to decrease expression of an upregulated nORF. The therapeutic polynucleotides of the disclosure, for example, may reduce the dysregulated nORF expression in a human subject. For example, the therapeutic agents of the disclosure may reduce dysregulated nORF expression e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, 98%, or 99%.

The expression level of the nORF expressed in a patient can be ascertained, for example, by evaluating the concentration or relative abundance of mRNA transcripts derived from transcription of the nORF. Additionally, or alternatively, expression can be determined by evaluating the concentration or relative abundance of the nORF following transcription and/or translation of an inhibitor that decreases an amount of the dysregulated nORF. Protein concentrations can also be assessed using functional assays, such as MDP detection assays. Expression can be evaluated by a number of methodologies known in the art, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of mRNAs.

### Nucleic Acid Detection

Nucleic acid-based methods for determining expression (e.g., of an RNA inhibitor or an RNA encoding the nORF) detection that may be used in conjunction with the compositions and methods described herein include imaging-based techniques (e.g., Northern blotting or Southern blotting). Such techniques may be performed using cells obtained from a patient following administration of the polynucleotide encoding the agent. Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, NY 11803-2500. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Detection techniques that may be used in conjunction with the compositions and methods described herein to evaluate nORF expression further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLID sequencing, and nanopore sequencing platforms. Additional methods of sequencing known in the art can also be used. For instance, expression at the mRNA level may be determined using RNA-Seq (e.g., as described in Mortazavi et al., Nat. Methods 5:621-628 (2008) the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Expression levels of the nORF may be determined using microarray-based platforms (e.g., single-nucleotide polymorphism arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and includes arrays fabricated by direct synthesis of oligonucleotides on a glass surface. Other systems may be used as known to one skilled in the art.

Amplification-based assays also can be used to measure the expression level of the nORF or RNA in a target cell following delivery to a patient. In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996), and in Heid et al., Genome Res. 6:986-994 (1996), the disclosures of each of which are incorporated herein by reference in their entirety. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Expression of the nORF can additionally be determined by measuring the concentration or relative abundance of a corresponding protein product (e.g., the nORF in a noncancerous cell or the dysregulated nORF). Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize expression of the nORF in a cell from a patient (e.g., a human patient) following delivery of the transgene encoding the nORF. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

### Pharmaceutical Compositions and Routes of Administration

The polynucleotides described herein may be formulated into a pharmaceutical composition for administration to a subject in a biologically compatible form suitable for administration in vivo. For example, the polynucleotides described herein may be administered in a suitable diluent, carrier, or excipient, and may further contain a preservative, e.g., to prevent the growth of microorganisms. Conventional procedures and ingredients for the selection and preparation of suitable compositions are described, for example, in Remington, J. P. The Science and Practice of Pharmacy, Easton, PA. Mack Publishers, 2012, 22nd ed. and in The United States Pharmacopeial Convention, The National Formulary, United States Pharmacopeial, 2015, USP 38 NF 33).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans to render the compositions suitable for administration to various animals is well understood, and the ordinarily skilled veterinary pharmacologist can design and/or perform such modification with merely ordinary, if any, experimentation.

The actual dosage amount of a composition of the present disclosure administered to a subject can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary.

The compositions utilized in the methods described herein can be administered to a subject by any suitable route of administration. For example, a composition containing an polynucleotide of the disclosure may be administered intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, intrathecally, orally, topically, locally, by inhalation, by injection, or by infusion (e.g., continuous infusion).

### Delivery of Polynucleotides

The delivery of a polynucleotide (e.g., oligonucleotide) of the invention to a cell e.g., a cell within a subject, such as a human subject e.g., a subject in need thereof, such as a subject having cancer can be achieved in a number of different ways. For example, delivery may be performed by contacting a cell with a polynucleotide of the invention either in vitro or in vivo. In vivo delivery may also be performed directly by administering a composition comprising a polynucleotide to a subject. These alternatives are discussed further below.

In general, any method of delivering a nucleic acid molecule (in vitro or in vivo) can be adapted for use with a polynucleotide of the invention (see e.g., Akhtar S. and Julian R L., (1992) Trends Cell. Biol. 2 (5): 139-144 and WO 1994/002595, which are incorporated herein by reference in their entireties). For in vivo delivery, factors to consider in order to deliver a polynucleotide molecule include, for example, biological stability of the delivered molecule, prevention of non-specific effects, and accumulation of the delivered molecule in the target tissue. The non-specific effects of a polynucleotide can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the oligonucleotide molecule to be administered.

For administering a polynucleotide systemically for the treatment of a disease, the polynucleotide can include alternative nucleobases, alternative sugar moieties, and/or alternative internucleoside linkages, or alternatively delivered using a drug delivery system; both methods act to prevent the rapid degradation of the polynucleotide by endo- and exo-nucleases in vivo. Modification of the polynucleotide or the pharmaceutical carrier can also permit targeting of the polynucleotide composition to the target tissue and avoid undesirable off-target effects. Polynucleotide molecules can be modified by chemical conjugation to lipophilic groups such as cholesterol to enhance cellular uptake and prevent degradation. In an alternative embodiment, the polynucleotide can be delivered using drug delivery systems such as a nanoparticle, a lipid nanoparticle, a polyplex nanoparticle, a lipoplex nanoparticle, a dendrimer, a polymer, liposomes, or a cationic delivery system. Positively charged cationic delivery systems facilitate binding of a polynucleotide molecule (negatively charged) and also enhance interactions at the negatively charged cell membrane to permit efficient uptake of a polynucleotide by the cell. Cationic lipids, dendrimers, or polymers can either be bound to a polynucleotide, or induced to form a vesicle or micelle that encases a polynucleotide. The formation of vesicles or micelles further prevents degradation of the polynucleotide when administered systemically. In general, any methods of delivery of nucleic acids known in the art may be adaptable to the delivery of the polynucleotides of the invention. Methods for making and administering cationic polynucleotide complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of polynucleotides include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, a polynucleotide forms a complex with cyclodextrin for systemic administration. Methods for administration and pharmaceutical compositions of polynucleotides and cyclodextrins can be found in U.S. Pat. No. 7,427,605, which is herein incorporated by reference in its entirety. In some embodiments the oligonucleotides of the invention are delivered by polyplex or lipoplex nanoparticles. Methods for administration and pharmaceutical compositions of oligonucleotides and polyplex nanoparticles and lipoplex nanoparticles can be found in U.S. Patent Application Nos. 2017/0121454; 2016/0369269; 2016/0279256; 2016/0251478; 2016/0230189; 2015/0335764; 2015/0307554; 2015/0174549; 2014/0342003; 2014/0135376; and 2013/0317086, which are herein incorporated by reference in their entirety.

### Liposomes and Membranous Delivery

The polynucleotides described herein can also be delivered using a variety of membranous molecular assembly delivery methods including polymeric, biodegradable microparticle, or microcapsule delivery devices known in the art. For example, a colloidal dispersion system may be used for targeted delivery of a polynucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide are delivered into the cell where the oligonucleotide can specifically bind to a target RNA and can mediate RNase H-mediated gene silencing. In some cases, the liposomes are also specifically targeted, e.g., to direct the oligonucleotide to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing a polynucleotide can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide preparation is then added to the micelles that include the lipid component. The cationic groups on the lipid interact with the oligonucleotide and condense around the oligonucleotide to form a liposome. After condensation, the detergent is removed, e.g., by dialysis, to yield a liposomal preparation of oligonucleotide.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable polynucleotide delivery vehicles, which incorporate a polynucleotide/cationic lipid complex as a structural component of the delivery vehicle, are further described in, e.g., WO 1996/037194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75:4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169). These methods are readily adapted to packaging oligonucleotide preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 1994/000569; WO 1993/024640; WO 1991/016024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417. Non-ionic liposomal systems have also been examined to determine their utility in the delivery of drugs to the skin, in particular systems comprising non-ionic surfactant and cholesterol. Non-ionic liposomal formulations comprising NOVASOME™ | (glyceryl dilaurate/cholesterol/polyoxyethylene-10-stearyl ether) and NOVASOME™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P.Pharma. Sci., 4 (6): 466).

Liposomes may also be sterically stabilized liposomes, comprising one or more specialized lipids that result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GM1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes comprising one or more glycolipids are known in the art. Papahadjopoulos et al. (Ann. N.Y. Acad. Sci., (1987), 507:64) reported the ability of monosialoganglio side GM1, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes comprising (1) sphingomyelin and (2) the ganglioside GM1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 1997/013499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides to macrophages.

Further advantages of liposomes include liposomes obtained from natural phospholipids are biocompatible and biodegradable; liposomes can incorporate a wide range of water and lipid soluble drugs; liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy) propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotide (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia) propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia) propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 1998/039359 and WO 1996/037194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer oligonucleotide into the skin. In some implementations, liposomes are used for delivering oligonucleotide to epidermal cells and also to enhance the penetration of oligonucleotide into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2,405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems comprising non-ionic surfactant and cholesterol. Non-ionic liposomal formulations comprising NOVASOME I (glyceryl dilaurate/cholesterol/polyoxyethylene-10-stearyl ether) and NOVASOME II (glyceryl distearate/cholesterol/polyoxyethylene-10-stearyl ether) may be used to deliver a drug into the dermis.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the present invention are described in PCT Publication Nos. WO 2009/088891, WO 2009/132131, and WO 2008/042973, which are hereby incorporated by reference in their entirety.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotides for use in the methods of the invention can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

### Lipid Nanoparticle-Based Delivery Methods

Polynucleotides of in the invention may be fully encapsulated in a lipid formulation, e.g., a lipid nanoparticle (LNP) or other nucleic acid-lipid particles. LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 2000/003683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 1996/040964.

In one embodiment, the lipid to drug ratio (mass/mass ratio) (e.g., lipid to oligonucleotide ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention. Non-limiting examples of cationic lipids include N,N-dioleyl-N,N-dimethylammonium chloride (DODAC), N,N-distearyl-N,N-dimethylammonium bromide (DDAB), N--(I-(2,3-dioleoyloxy) propyl)-N,N,N-trimethylammonium chloride (DOTAP), N--(I-(2,3-dioleyloxy) propyl)-N,N,N-trimethylammonium chloride (DOTMA), N,N-dimethyl-2,3-dioleyloxy) propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), 1,2-Dilinoleoyl-3-trimethylaminopropane chloride salt (DLin-TAP.CI), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino) ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyetetrahydro--3aH-cyclopenta[d][1,3]dioxol-5-amine (ALN100), (6Z,9Z,28Z,31Z)-heptatriaconta-6,9,28,31-tetraen-19-yl4-(dimethylamino) bu-tanoate (MC3), 1,1′-(2-(4-(2-((2-(bis(2-hydroxydodecyl)amino)ethyl) (2-hydroxydodecyl) ami-no)ethyl) piperazin-1-yeethylazanediyedidodecan-2-ol (Tech G1), or a mixture thereof. The cationic lipid can comprise, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 60 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C12), a PEG-dimyristyloxypropyl (C14), a PEG-dipalmityloxypropyl (C16), or a PEG-distearyloxypropyl (C18). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 50 mol % of the total lipid present in the particle.

### Examples

The following examples further illustrate the invention but should not be construed as in any way limiting its scope.

### Example 1. SiRNA Knockdown Assays

### Materials

Hs578T (HTB-126) and SCC25 (CRL-1628), Dulbecco's Modified Eagle's Medium (DMEM; 30-2002), Dulbecco's modified Eagle's Medium: Ham's F12 medium (DMEM/F12; 30-2006), Fetal bovine serum (FBS; 30-2020) from ATCC, human insulin (12585-014) and Trypsin-EDTA 0.25% (25200-056) from Gibco, hydrocortisone (A16292-03), Lipofectamine RNAiMax Reagent (13778-030), OptiMEM Reduced Serum Medium (OptiMEM, 31985088), SuperScript IV VILO Master Mix (11766050), Hoechst 33342 (H1399), Propidium Iodide (J66584-AB) and custom Taqman assays for Target 1 and Target 3 from Thermo Fisher Scientific, Dicer-Substrate Short Interfering RNAs (DsiRNAs) for Target 1, Target 3, HPRT-S1 (51-01-08-02) & Negative Control (51-01-14-03), Human HPRT qPCR Assay (51-01-08-04) and Human GAPDH qPCR Assay (Hs.PT.39a.22214836) from Integrated DNA Technologies, RNeasy Plus Mini Kit (74134) from Qiagen and LUNA® Universal Probe qPCR Master Mix (M3004) from New England BioLabs were utilized in all experiments.
ENST00000455557.2 (SEQ ID NO: 1)=Target 1
ENST00000383686.3 (SEQ ID NO: 3)=Target 3

Dicer-substrate siRNAs (DsiRNAs) having the following sequences were ordered from IDT.

Target 1: nucleotides 130-597 of SEQ ID NO: 1
(siRNA targets bolded and underlined)
ATGTTAGCCAGGATAGTTTCAATTTCCGGACCTCGTGATCCGCCCGCCTC
GGCCTCCCAAAGTGCTGGGATTATAGGCGTGAGCCACCGCGCCCAGCCCC
TTTCATTTTTGATATGGAGTTTTGTTCTTGTTGCCCAGGCTGGAATGCAA
TGGCGCGATCTCAGCTCACTGCAACCTCTGCCTCCTGGGTTCAAGCGATT
CTCCTGGTTCAGCCTCCTGAGTAGCTGGGATTACAGGAATGCGCCACCAC
ACCTGGCTACTTTTGTATTTTTAGTAGAGATGGGGTTTCTCCACGTGCAT
CAGGCTAGTCTCACACTCCCGACTTCAGATGATCCACCCGCCTCGGCCTC
CCAAAGTGCTGGGATTACAGGCGTGAGCCACCGTGCCTGGCCAAAGCCTT
CTTCACTTTCATTTTGTGGCTGCTGTTGTGAAATCTGCAGAACCTCAAGC
TGTTTGGACTCCATCTTC

TABLE 2
Target 1 siRNA sequences

| Target | SEQ ID NO | Sequence 1 | SEQ ID NO: | Sequence 2 | SEQ ID NO: |
|---|---|---|---|---|---|
| GGCCAAAGCCTT | SEQ ID NO: 5 | rGrGrCrCrArArAr | SEQ ID NO: 6 | rArArUrGrArArAr | SEQ ID NO: 7 |
| CTTCACTTTCAT | | GrCrCrUrUrCrUr | | GrUrGrArArGrAr | |
| T | | UrCrArCrUrUrUr | | ArGrGrCrUrUrUr | |
| | | CrATT | | GrGrCrCrArG | |
| CTACTTTTGTAT | SEQ ID NO: 8 | rCrUrArCrUrUrUr | SEQ ID NO: 9 | rArUrCrUrCrUrAr | SEQ ID NO: 10 |
| TTTTAGTAGAGA | | UrGrUrArUrUrUr | | CrUrArArArArAr | |
| T | | UrUrArGrUrArGr | | UrArCrArArArAr | |
| | | ArGAT | | GrUrArGrCrC | |
| CGATCTCAGCTC | SEQ ID NO: 11 | rCrGrArUrCrUrC | SEQ ID NO: 12 | rCrArGrArGrGrU | SEQ ID NO: 13 |
| ACTGCAACCTCT | | rArGrCrUrCrArCr | | rUrGrCrArGrUrG | |
| G | | UrGrCrArArCrCr | | rArGrCrUrGrArG | |
| | | UrCTG | | rArUrCrGrCrG | |

Target 3: nucleotides 115-711 of SEQ ID NO: 3
(siRNA targets bolded and underlined)
ATGCTTCCAGTCCAGAGGAGAAAGCTAGACCCACTCCTCAAAAAGTATAG
GCATCATAAGAAGGCCACAAGGACAAAAAGAAGGAGGAAGGAGAAAATGG
AGGCCCAGTGCTCCCCTGTTCCTCCAACACCTTCAACACCACCACAAAGT
GAAGAAGATGAGGCTGTAGACAAAAAGCCAACTCTACTCAGTGCCCAGGA
AGATACTCCTGACCTTCTCCATGAGGACAGATTACAGTATCTGCAGGAAG
AGGGTTCCGGTGTGATGCATCAGGAATGTCAGATCCAGTCCTGTGAGCTC
TCGGTGGCTCAGAAGCCCAGGCCCTCTTCTCCTGCAGTGACATCCTTGGC
ATCACCACCACTCTGCTTTGGCAGTTTCCTAAGCTGTGTCTGCCAGACCT
TCTCAAGGTCTAGGAAGCAGAAGCCTCCCAGAAGAAAGGGTAACAACCAG
GCTGAGGCAGGAGGTGATGCTGAGGTCCTGAGACCTGGCCCGGCAAAACC
AGAGTTGAGCCTCAGCAAAACGTGTAGCCACTTGAAATCGATAGAGCTTT
CCTTTGTGTTTAGTTTTATAGTTCTCTCTGTATGTCATTGTTCCTCT

TABLE 3
Target 3 siRNA sequences

| Target | SEQ ID NO | Sequence 1 | SEQ ID NO: | Sequence 2 | SEQ ID NO: |
|---|---|---|---|---|---|
| CCATGAGGACAG | SEQ ID NO: 14 | rCrCrArUrGrArGrG | SEQ ID NO: 15 | rCrArGrArUrArCrU | SEQ ID NO: 16 |
| ATTACAGTATCT | | rArCrArGrArUrUrA | | rGrUrArArUrCrUrG | |
| G | | rCrArGrUrArUrCT | | rUrCrCrUrCrArUrG | |
| | | G | | rGrArG | |
| CATCAGGAATGT | SEQ ID NO: 17 | CrArUrCrArGrGrA | SEQ ID NO: 18 | rArGrGrArCrUrGr | SEQ ID NO: 19 |
| CAGATCCAGTCC | | rArUrGrUrCrArGrA | | GrArUrCrUrGrArCr | |
| T | | rUrCrCrArGrUrCC | | ArUrUrCrCrUrGrAr | |
| | | T | | UrGrCrA | |
| ACTTGAAATCGA | SEQ ID NO: 20 | rArCrUrUrGrArArA | SEQ ID NO: 21 | rArArGrGrArArArG | SEQ ID NO: 22 |
| TAGAGCTTTCCT | | rUrCrGrArUrArGrA | | rCrUrCrUrArUrCrG | |
| T | | rGrCrUrUrUrCrCT | | rArUrUrUrCrArArG | |
| | | T | | rUrGrG | |

The three DsiRNAs for Target 1 or Target 3 were pooled for experiments.

### Cell Culture

Hs578T cells were cultured and maintained in DMEM medium with 10% FBS and 0.01 mg/ml insulin with subculturing at a split ratio of 1:5 every 4 days. SCC25 cells were cultured and maintained in DMEM/F12 medium with 10% FBS and 400 ng/ml hydrocortisone with subculturing at a split ratio of 1:10 every 4 days.

### DsiRNA Transfection

DsiRNAs were resuspended according to manufacturer's protocol. Briefly, individual Target DsiRNAs were resuspended in nuclease-free water to 75,000 nM. DsiRNAs specific to Target 1 or Target 3 were then pooled to a final individual concentration of 25,000 nM. HPRT-S1 and Negative Control DsiRNA were solubilized to 25,000 nM in nuclease-free water.

Lipofectamine/DsiRNA transfection solutions were prepared at 10× final concentration by first diluting Lipofectamine RNAiMax 25-fold in OptiMEM base medium and individual DsiRNAs or DsiRNA pools were diluted to 500 nM in OptiMEM base medium. Each diluted DsiRNA or DsiRNA pool was mixed with an equal volume of diluted Lipofectamine RNAiMax and incubated at ambient temperature for 20 minutes. Transfection solutions were then plated into individual wells of 6- or 24-well tissue culture plates at 200 uL or 50 uL respectively. Hs578T or SCC25 cells diluted to 200,000 cells/mL in OptiMEM Reduced Serum medium containing 5% FBS were then added to the wells containing DsiRNA transfection solutions, 2000 μL/well for 6-well plates and 500 μL/well for 24-well plates. Transfections were incubated for 48 hours at 37° C., 5% CO₂.

Real-Time qPCR

Total RNA isolated from Hs578T or SCC25 cells was extracted using RNeasy Plus kit (Qiagen) according to the manufacturer's directions and then cDNA was prepared using the SuperScript IV VILO cDNA synthesis kit. Levels of HPRT1, Target1 and Target3 mRNA expression were assessed by real-time qPCR using the StepOne Plus Instrument and Luna Universal Probe qPCR Master Mix, normalized with the GAPDH housekeeping control expression. Real-time qPCR analysis was carried out using the 2(-Delta Delta C (T)) method.

### Cell Proliferation

DsiRNA-transfected Hs578T or SCC25 cells from 6-well plates were collected by removing the transfection media and washing once with 1000 uL of Phosphate Buffered Saline (PBS). After removal of PBS, 500 uL of Trypsin-EDTA was added and incubated for 5 minutes at 37° C., 5% CO₂. 1500 uL of the appropriate culture media was added to the wells, mixed 3 times and solution transferred to a sterile 15-mL centrifuge tube. Cells were pelleted by centrifugation at 150 g or 5 minutes, supernatant removed. All DsiRNA-treatment groups for Hs578T were resuspended to 25,000 cells/mL in DMEM with 10% FBS and 0.01 mg/mL insulin followed by plating into 16 wells of a clear bottom 96-well tissue culture plate at 50 μL/well. An additional 75 ul of DMEM growth media was added to the plate after all treatment groups were plated. All DsiRNA-treatment groups for SCC25 were resuspended to 30,000 cells/mL in DMEM/F12 with 10% FBS and 400 ng/ml insulin followed by plating into 16 wells of a clear bottom 96-well tissue culture plate at 50 μL/well. An additional 75 ul of DMEM growth media was added to the plate after all treatment groups were plated. Cell plates were incubated at 37° C., 5% CO₂. After 16 hours of incubation, 2 wells of each treatment group were stained with the nuclear dyes Hoechst 33342 and Propidium Iodide by adding 25 ul of DMEM containing 48 uM Hoechst 33342 and 9 uM Propidium Iodide. Plates were placed back in the incubator for 30 minutes and then read on an SPT Labtech Mirrorball Laser Scanning Cytometer. Whole well fluorescent images at 405 nm excitation/460 nm emission, Hoechst 33342 live cell fluorescence, and 488 nm excitation/560 nm emission, Propidium Iodide dead/dying cell fluorescence, were collected and used for cell viability at T=0 hours. Plates were then placed back in the incubator and incubated for an additional 96-Hours. At 96-hours the remaining wells of the plates were stained with the nuclear dyes Hoechst 33342 and Propidium Iodide by adding 25 ul of DMEM containing 48 uM Hoechst 33342 and 9 uM Propidium Iodide. Plates were placed back in the incubator for 30 minutes and then read on an SPT Labtech Mirrorball Laser Scanning Cytometer. Whole well fluorescent images were then used to determine the growth rate and overall cell viability for each of the DsiRNA-treatment groups.

### Results

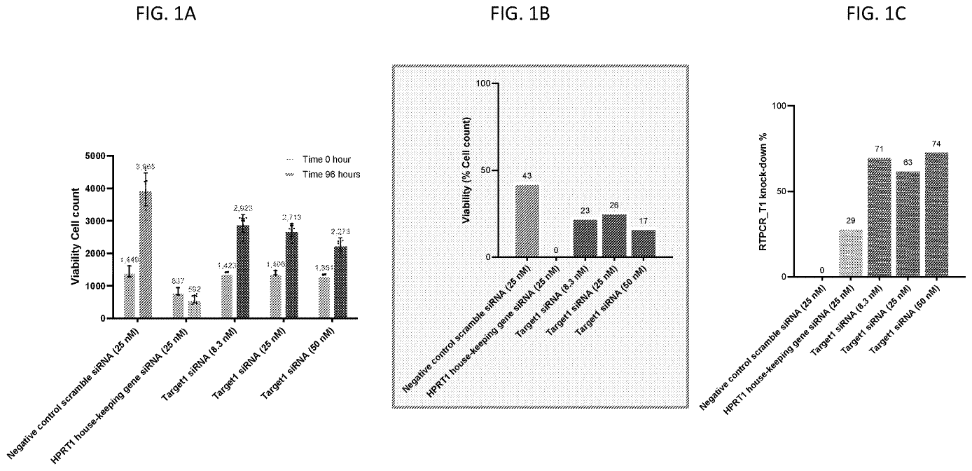

siRNA knockdown of ENST00000455557.2 (SEQ ID NO: 1) in a head and neck cancer cell line (SCC25) was assessed. FIG. 1A shows viable cell counts in the presence of a scrambled siRNA (negative control), a positive control (HPRT1), and increasing concentrations of target siRNA. FIG. 1B shows % viable cell counts normalized to time 0 hours in the presence of a scrambled siRNA, a positive control (HPRT1), and increasing concentrations of target siRNA. FIG. 1C shows % knockdown in the presence of a scrambled siRNA (negative control), a positive control (HPRT1), and increasing concentrations of target siRNA. HPRT1 house-keeping siRNA showed 0% cell viability after 96 hours, and negative control scramble-siRNA showed 43% cell viability after 96 hours. Target specific siRNA showed 26% cell viability after 96 hours.

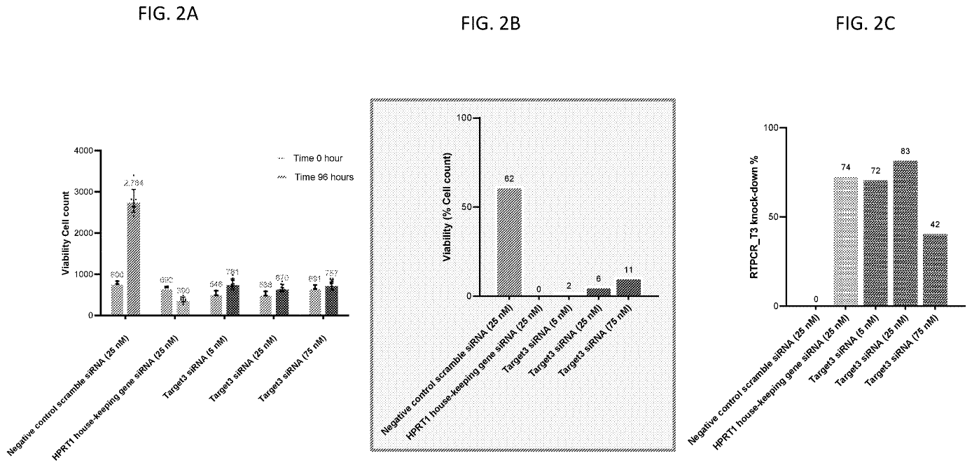

siRNA knockdown of ENST00000383686.3 (SEQ ID NO: 3) in a breast cancer cell line (Hs578T) was assessed. FIGS. 2A-2C are graphs showing siRNA knockdown of ENST00000383686.3 (SEQ ID NO: 3) in a breast cancer cell line (Hs578T). FIG. 2A shows viable cell counts in the presence of a scrambled siRNA (negative control), a positive control (HPRT1), and increasing concentrations of target siRNA. FIG. 2B shows % viable cell counts normalized to time 0 hours in the presence of a scrambled siRNA, a positive control (HPRT1), and increasing concentrations of target siRNA. FIG. 2C shows % knockdown in the presence of a scrambled siRNA (negative control), a positive control (HPRT1), and increasing concentrations of target siRNA. HPRT1 house-keeping siRNA showed 0% cell viability after 96 hours, and negative control scramble-siRNA showed 462% cell viability after 96 hours. Target specific siRNA showed 6% cell viability after 96 hours.

### Example 2. Characterization of Target 1 and Target 3 Knockdown

This study was undertaken to further characterize the function of two protein targets, Target 1 and Target 3, shown to be expressed and susceptible to mRNA knock-down by DsiRNA (Dicer-substrate small interfering RNA) probes in SCC25 and Hs578T cells respectively as shown in Example 1. Two other targets, Target 2 and Target 5, were also analyzed for comparison. This study was designed to generate clear, in-depth data to determine cell-specific downregulation effects of Target 1 and Target 3 on cancer cell viability and proliferation and evaluate protein localization of tagged Target 1 and Target 3 by overexpression.

For Target 1, following successful knockdown of Target 1 (>70%) in SCC25 cells using both DsiRNA and ASO (antisense oligonucleotides) pools, the dose dependent effect of Target 1 knockdown by DsiRNAs and ASOs on cell viability and proliferation over 144-hours was evaluated. It was observed that knockdown by both DsiRNAs and ASOs resulted in a dramatic decrease (50%) in viable cells post transfection at all doses versus the comparable dose of negative scramble control (NC). A dose dependent knockdown of viable SCC25 cells was observed at all timepoints using DsiRNA while a dose dependent decrease in viable SCC25 cells using ASO treatment was only observed at the T=0. These results indicated that expression of Target 1 has a functional consequence on the viabilty of the SCC25 tumor line. Target 1 knockdown was additionally evaluated in Hs578T cells as a counter screen and resulted in a significant growth suppression even though expression data indicate that Target 1 was not observed to be expressed in Hs578T and may not impact cell function.

For Target 3, following successful knockdown of Target 3 (>70%) in Hs578T cells using both DsiRNA and ASO pools, the dose dependent effect of Target 3 knockdown by DsiRNAs and ASOs on Hs578T viability and proliferation over 96-hours was evaluated. It was observed that knockdown by both DsiRNAs and ASOs resulted in a decrease in viable cells post transfection at all doses versus the comparable dose of negative scramble control (NC). Additionally, multiple doses of Target 3 probe pools suppressed cell growth between 0 to 96 hour timepoints versus NC transfection with the multiple doses showing complete growth suppression over four days. These results indicate that expression of Target 3 has a functional consequence on the viabilty and growth of the Hs578T tumor line in culture. Target 3 knockdown was additionally evaluated in SCC25 cells as a counter screen and was shown to decrease viable cells post transfection which could be attributed to a moderate level of Target 3 expression in SCC25.

Evaluation of target expression in HEK293T cells proved challenging as low initial transfection efficiency and poor adherence of HEK293T cells during the immunofluorescence staining process made imaging challenging. Improved adherence of transfected cells was achieved using poly-d-lysine resulting in less than 25% of cells lost during staining, allowing detection of four targets by FLAG-tag immunofluorescent staining. Detection of localized expression of targets proved difficult as the majority of staining was diffuse throughout the cells.

### Results

Prior to beginning target knockdown and overexpression work, SCC25, Hs578T and HEK293T cells were analyzed for expression of the original four protein targets from Example 1 using a Taqman single-plex assay format. Surprisingly, SCC25 showed reasonable expression of Target 5 and Target 3 in addition to Target 1 while Hs578T cells expressed Target 5 in addition to Target 3. HEK293T cells poorly expressed all but Target 3, Table 4. Both SCC25 and Hs578T could be useful cell models for the future assessment of Target 5 function in knockdown studies which was paused due to low abundance in the original four cell lines: HCT116, MCF7, SNU1 and T84.

TABLE 4
Re-evaluation of Target Expression in tumor cell models and HEK293Ts.

| | C_{T} | | | | | | |
|---|---|---|---|---|---|---|---|
| | HCT116 | | | T84 | SCC25 | | HEK293T |
| | (Colon/ | MCF7 | SNU1 | (Colon/ | (Squamous | Hs578T | (Human |
| Target | colorectal | (Breast | (Stomach | colorectal | cell | (Breast | embryonic |
| Name | cancer) | cancer) | cancer) | cancer) | carcinoma) | cancer) | kidney) |
| GAPDH | 15.5 | 16.3 | 14.6 | 14.4 | 21.1 | 21.2 | 22.4 |
| Target 1 | 30.4 | >45.0 | >45.0 | 39.1 | 30.9 | 39.8 | 40.6 |
| Target 2 | 34.8 | 43.5 | 41.4 | 35.7 | 39.8 | 38.9 | >45.0 |
| Target 3 | 32.7 | 31.0 | 37.9 | 32.5 | 33.7 | 29.5 | 32.9 |
| Target 5 | 38.1 | 37.0 | 41.0 | 33.6 | 28.6 | 29.8 | 36.5 |
| HPRT1 | 24.7 | 25.6 | 25.0 | >45.0 | not tested | not tested | not tested |

| | |
|---|---|
| Suitable for Knock-down | Potential for Knock-down |
| Studies | Studies |
| CT level < 32 | CT between 32-35 |

### Knockdown and Functional Consequence of Target 1 in SCC25 Cells

### Target 1 Knockdown Protocol

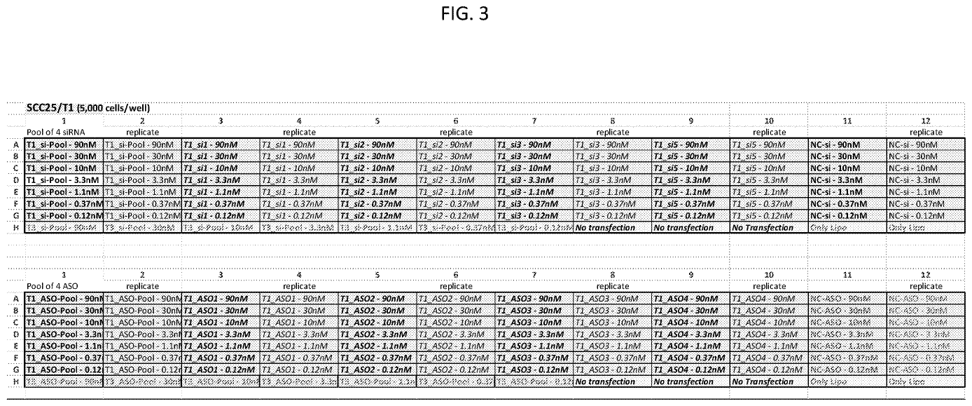

After validation of DsiRNA and ASO probe dose range, transfection solutions were prepared and plated into individual wells of five 96-well tissue culture plates, plates correspond to post-transfection timepoints of 0, 24, 48, 96, and 144 hours. 150 μL of SCC25 cells in OptiMEM reduced serum medium with 5% FBS (33,333 cells/mL) was added to the plates and incubated for 48 hours at 37° C., 5% CO₂. After 48 hours, the transfection media was aspirated off, replaced with 200 μL of DMEM growth media and the plates returned to a 37° C., 5% CO₂ incubator. At each timepoint, a plate was removed from the incubator, growth media removed and 100 μL of PBS containing the nuclear dyes Hoechst 33342 (8 UM) and Propidium Iodide (1.5 UM) added, incubated for 30 minutes and read on the Mirrorball Imaging Cytometer. SCC25 Platemap showing the layout of DsiRNA and ASO probe pools for SCC25 transfection is shown in FIG. 3.

### Consequence of Target 1 Knockdown on SCC25 Growth and Viability by DsiRNA.

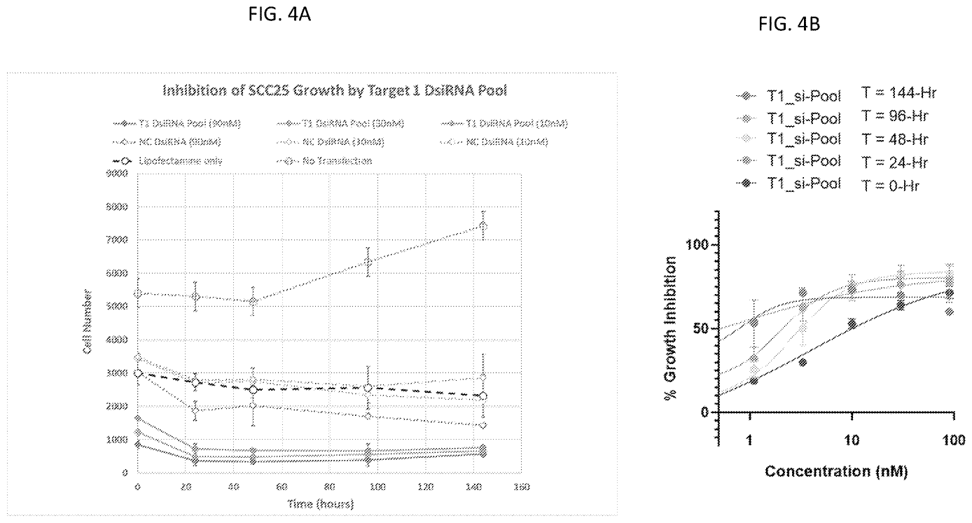

As seen in FIG. 4A, DsiRNA knockdown of Target 1 at all doses resulted in a dramatic decrease (50%) in viable cells post transfection versus the comparable dose of negative scramble control (NC). The knockdown of Target 1 appeared to suppress cell growth between the 24 to 144 hour timepoints versus no transfection but NC transfection also appeared to suppress the cell growth during these time points, so no conclusion can be drawn about growth inhibition. The cells appeared to be intolerable of the transfection conditions as LIPOFECTAMINE™ 2000 alone caused a decrease in cell viability of a similar magnitude (>45%) to the NC control. The DsiRNA pool also showed a dose dependent decrease in the number of viable cells at each timepoint collected (FIG. 4B) indicating that expression of Target 1 has a functional consequence on the viabilty of the SCC25 tumor line.

### Consequence of Target 1 Knockdown of SCC25 Growth and Viability by ASOs.

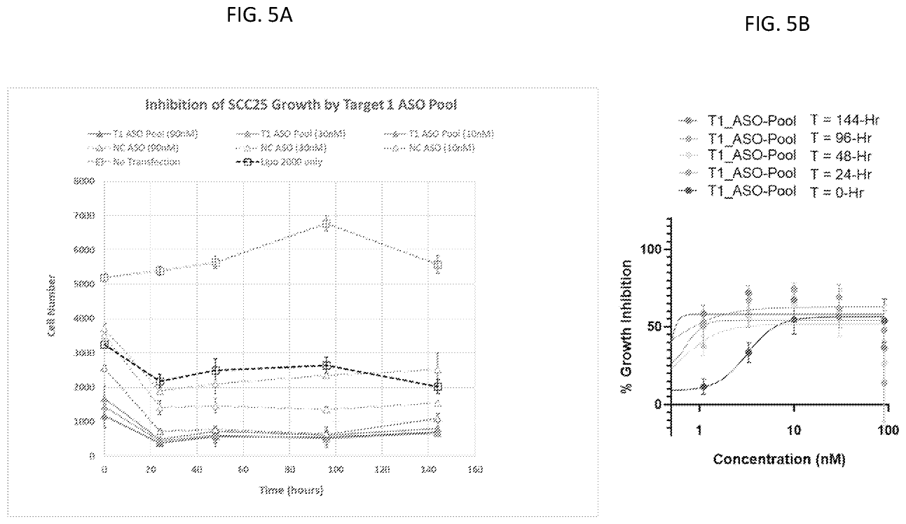

As seen in FIG. 5A, ASO knockdown of Target 1 at all doses resulted in a dramatic decrease (50%) in viable cells post transfection versus the comparable dose of negative scramble control (NC). The cell viability continued to drop at 24 hours post transfection but NC transfection at all doses demonstrated an even larger drop in viability between 0 and 24 hours making it difficult to ascertain the consequence of Target 1 knockdown at later timepoints using ASOs. The cells again appeared to be intolerable of the transfection conditions as LIPOFECTAMINE™ 2000 alone caused a decrease in cell viability of a similar magnitude (>40%) to the NC control. The dose dependence of ASO treatment by viable cell count could only be observed at the T=0 timepoint, FIG. 5B.

### Counter-Screen: Consequence of Target 1 Knockdown of Hs578T Growth and Viability by DsiRNA and ASOs.

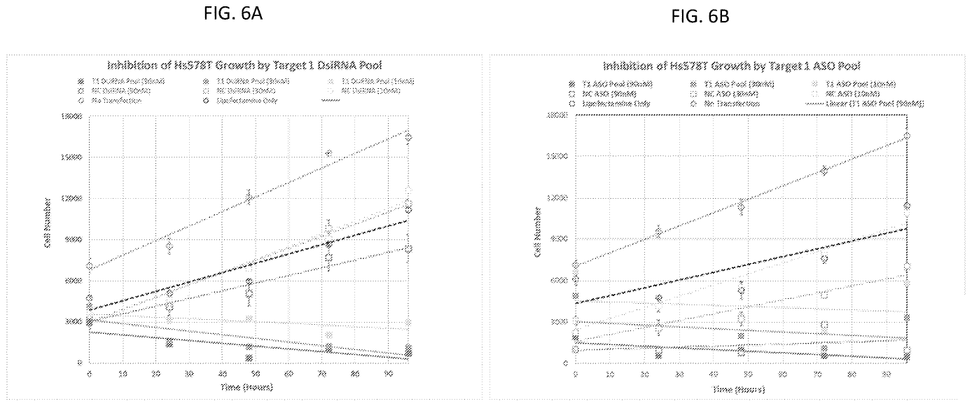

Target 1 was evaluated in Hs578T by DsiRNA and ASO pool knockdown. As seen in FIGS. 6A and 6B, Target 1 knockdown by DsiRNA (FIG. 6A) resulted in a significant suppression of growth relative to the comparable dose of negative scramble control (NC). Target 1 knockdown by ASO (FIG. 6B) also resulted in a significant suppression of growth but, surprisingly, Target 1 ASO treated cells had higher viability relative to the comparable dose of negative scramble control (NC) immediately post-transfection. The results for Target 1 knockdown in Hs578T cells were surprising as expression data indicate that Target 1 was not observed to be expressed in Hs578T and should not impact cell function. Perhaps off-target activity of both the DsiRNA and ASO probes could result in the observed growth inhibition and should be evaluated.

### Knockdown and Functional Consequence of Target 3 in Hs578T Cells

### Target 3 Knockdown Protocol.

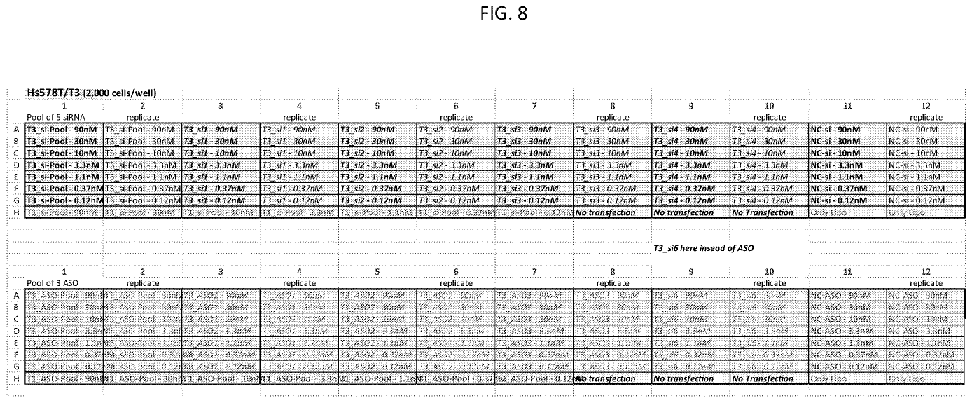

After validation of DsiRNA and ASO probe dose range, transfection solutions were prepared and plated into individual wells of five 96-well tissue culture plates, plates correspond to post transfection timepoints of 0, 24, 48, 72, and 96 hours. 150 UL of Hs578T cells in OptiMEM reduced serum medium with 5% FBS (13,333 cells/mL) was added to the plates and incubated for 48 hours at 37° C., 5% CO₂. After 48 hours, the transfection media was aspirated off, replaced with 200 μL of DMEM growth media, and the plates were returned to a 37° C., 5% CO₂ incubator. At each timepoint, the plate was removed from the incubator, growth media was removed, and 100 μL of PBS containing the nuclear dyes Hoechst 33342 (8 μM) and Propidium Iodide (1.5 μM) added, incubated for 30 minutes and read on the Mirrorball Imaging Cytometer. Hs578T Platemap and the layout of DsiRNA and ASO probe pools for Hs578T transfection is shown in FIG. 8.

### Consequence of Target 3 Knockdown of Hs578T Growth and Viability by DsiRNA.

As seen in FIG. 8A, DsiRNA knockdown of Target 3 at all doses resulted in a decrease in viable cells post transfection versus the comparable dose of negative scramble control (NC). Knockdown of Target 3 at all doses was able to suppress cell growth between from 0 to 96 hours versus NC transfection with the 90 nM dose showing nearly complete growth suppression over 4 days. Transfection with LIPOFECTAMINE™ alone was detrimental to cell viability reducing viable cells by 33% post transfection, similar to the NC control. Additionally, the dose dependency of the DsiRNA pool on the number of viable cells improved with time post-transfection (FIG. 8B) indicating that expression of Target 3 has a functional consequence on the viabilty and growth of the Hs578T tumor line in culture.

### Consequence of Target 3 Knockdown of Hs578T Growth and Viability by ASOs.

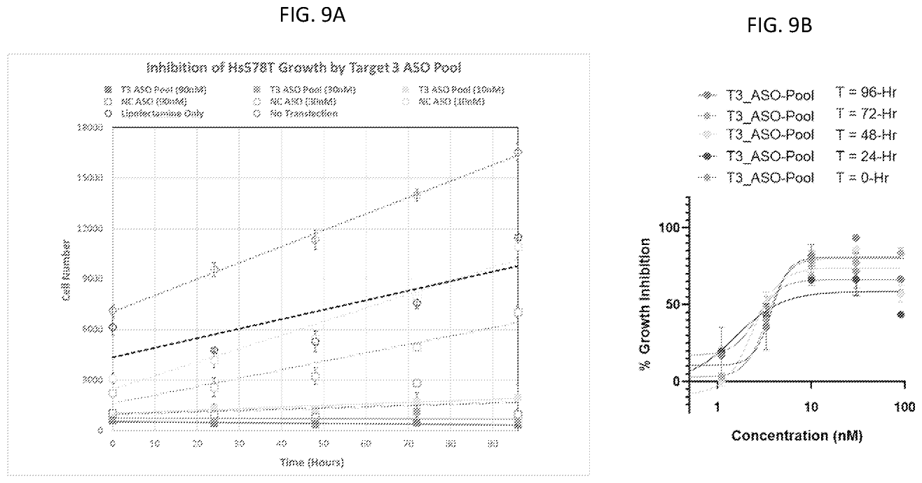

As seen in FIG. 9A, ASO knockdown of Target 3 at all doses resulted in a decrease in viable cells post transfection versus the comparable dose of negative scramble control (NC). Knockdown of Target 3 at all doses was able to suppress cell growth between from 0 to 96 hours vs NC transfection with nearly complete growth suppression down to 10 nM dose over 4 days. Transfection with LIPOFECTAMINE™ 2000 alone was again detrimental to cell viability reducing viable cells by 33% post transfection, similar to the NC control. Additionally, the does dependency of the DsiRNA pool on the number of viable cells was maintained from 48 to 96 hours post-transfection (FIG. 9B) indicating that expression of Target 3 has a functional consequence on the viabilty and growth of the Hs578T tumor line in culture.

### Counter-Screen: Consequence of Target 3 Knockdown of SCC25 Growth and Viability by DsiRNA and ASOs.

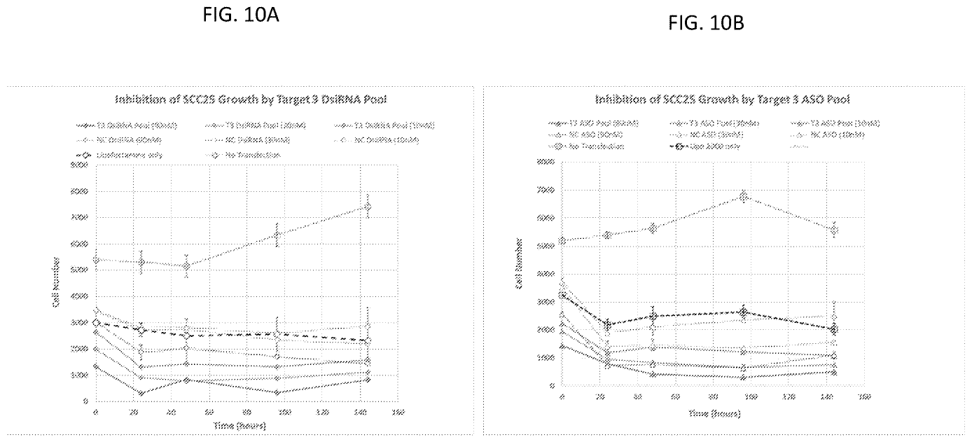

Target 3 was evaluated in SCC25 by DsiRNA and ASO pool knockdown. As seen in FIGS. 10A and 10B, Target 3 knockdown by DsiRNA (FIG. 10A) and ASO (FIG. 10B) resulted in a decrease in viable cells post transfection versus the comparable dose of negative scramble control (NC). Although the effect of Target 3 on SCC25 cell viability was unexpected, the earlier observation that Target 3 is expressed at a reasonable level in SCC25 could implicate its involvement in SCC25 viability.

### Target 1 and Target 3 Cell Transfection and Immunofluorescence Characterization

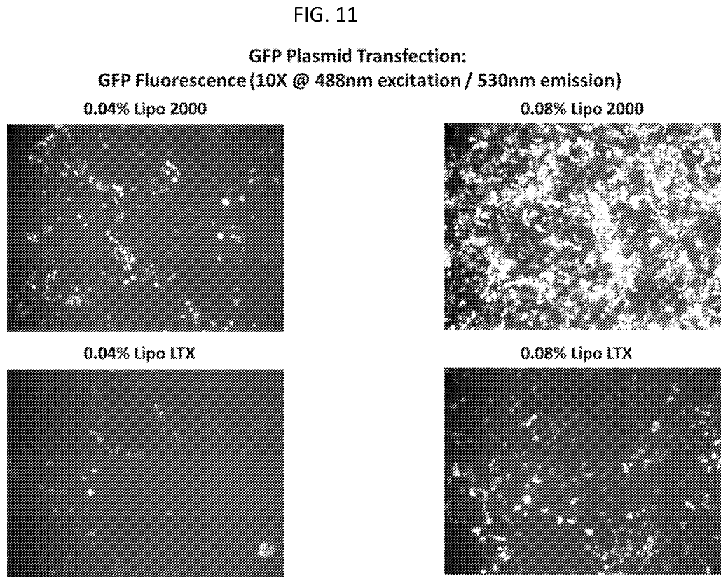

Initial studies focused on the optimization of transfection conditions using GFP and found LIPOFECTAMINE™ 2000 at 0.08% yielded the highest percentage of transfected cells and were the conditions used for the remainder of the study, FIG. 11.

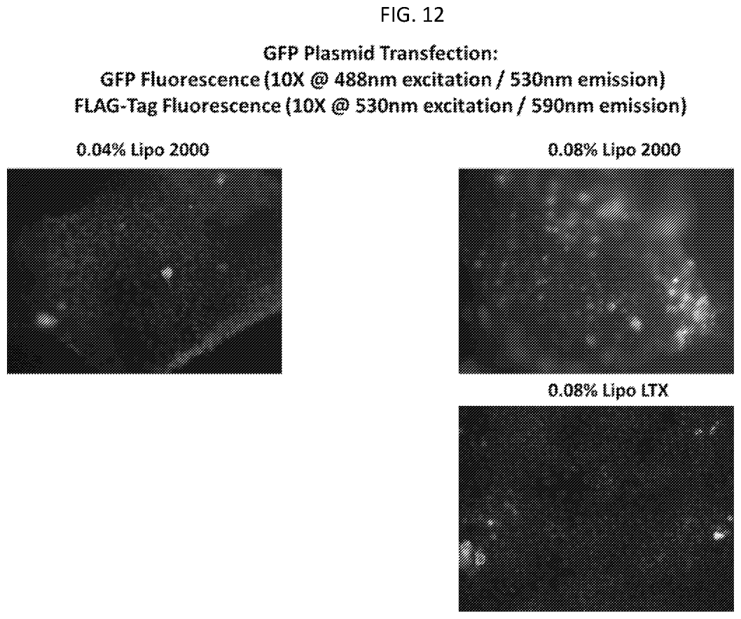

Follow-on studies focused on detecting the FLAG protein tag on overexpressed GFP and the 4 targets by immunofluorescence. Immunofluorescence showed that overexpressed GFP could be detected using the AF594-labelled FLAG antibody from CST and corroborated the use of 0.08% LIPOFECTAMINE™ 2000 but the number of transfected cells detected by GFP fluorescence was 2-3× higher than detected by the FLAG-tag antibody, FIG. 12. Immunofluorescence staining revealed that HEK293T cells did not adhere well to the tissue culture plate during the multi-step immunofluorescence protocol which affects identification of overexpressed cells.

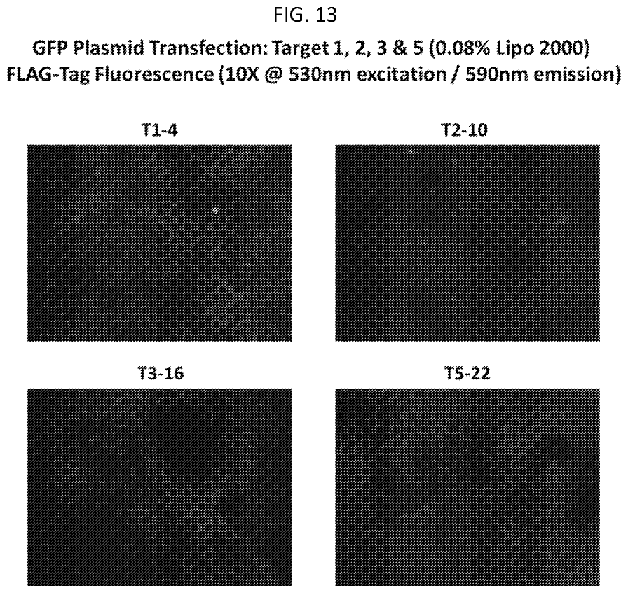

Immunofluorescence of overexpressed targets by FLAG-tag immunofluorescence proved challenging and initially only showed that expression of Target 1 (T1-4) and Target 2 (T2-10) could be detected and the percentage of cells transfected was very low, FIG. 13.

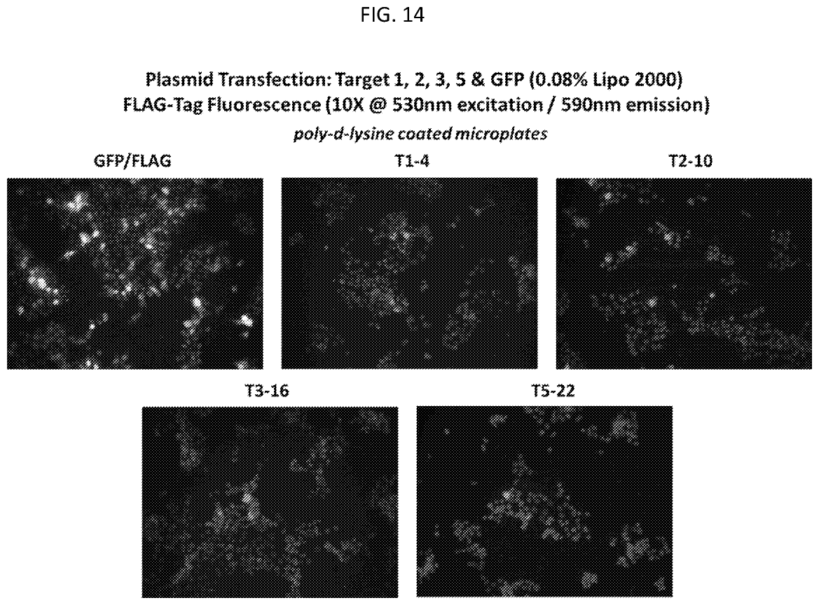

In order to improve the detection of targets, several coatings were evaluated to improve the adherence of cells in the wells of the microplate such that they would survive the multistep immunofluorescence staining procedure. After several rounds of testing, it was shown that coating the microplate wells with a layer of poly-d-lysine improved adhesion of the HEK293T cells such that less than 25% of cells were lost during staining (FIG. 14). As also seen in FIG. 14, the improvements in cell plating and transfection allowed the detection of all 4 targets by FLAG-tag immunofluorescent staining.

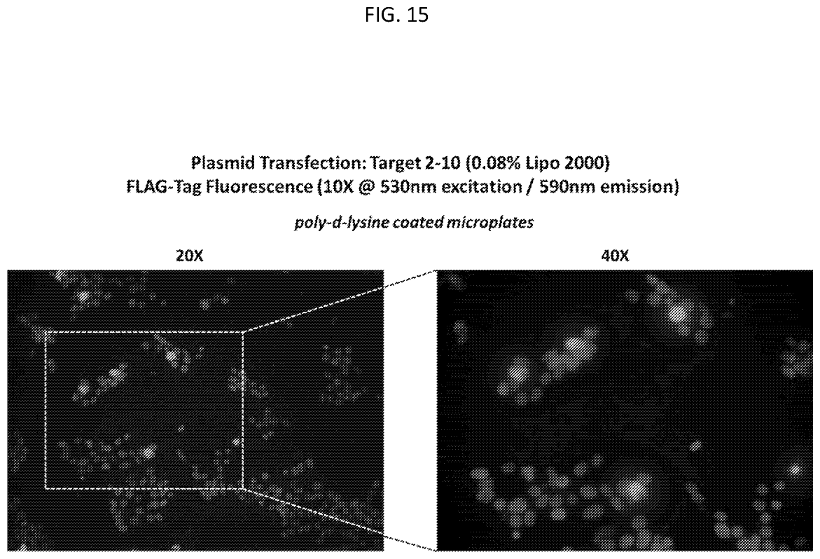

Detection of localized expression of targets proved difficult as the majority of staining was diffuse throughout the cell. Evaluation of Target 2 expression, as an example, showed diffuse staining of the cells at higher magnification and no localization within the cell, FIG. 15.

### Methods and Materials

### Target 1 Knockdown of SCC25 Growth and Viability by DsiRNA

Materials. SCC25 (CRL-1628), Dulbecco's modified Eagle's Medium: Ham's F12 medium (DMEM/F12; 30-2006), Fetal bovine serum (FBS; 30-2020) from ATCC, human insulin (12585-014) and Trypsin-EDTA 0.25% (25200-056) from Gibco, hydrocortisone (A16292-03), LIPOFECTAMINE™ 2000 Reagent (11668027), OptiMEM Reduced Serum Medium (OptiMEM, 31985088), SuperScript IV VILO Master Mix (11766050), Hoechst 33342 (H1399), Propidium Iodide (J66584-AB) and custom Taqman assays for Target 1 and Target 3 from Thermo Fisher Scientific, Dicer-Substrate Short Interfering RNAs (DsiRNAs) for Target 1, Target 3, HPRT-S1 (51-01-08-02), and Negative Control (51-01-14-03), Human HPRT qPCR Assay (51-01-08-04) and Human GAPDH qPCR Assay (Hs.PT.39a.22214836) from Integrated DNA Technologies, RNeasy Plus Mini Kit (74134) from Qiagen and LUNA® Universal Probe qPCR Master Mix (M3004) from New England BioLabs were utilized in all experiments.

Cell Culture: SCC25 cells were cultured and maintained in DMEM/F12 medium with 10% FBS and 400 ng/ml hydrocortisone with subculturing at a split ratio of 1:10 every 4 days.

DsiRNA Transfection: DsiRNAs stock solutions for Target 1 probes were resuspended according to manufacturer's protocol; T1-1, T1-2, T1-3, and T1-5. Briefly, individual Target DsiRNAs were resuspended in nuclease-free water to 75,000 nM. Negative Control DsiRNA were solubilized to 25,000 nM in nuclease-free water.

Working solutions of individual DsiRNAs and negative control DsiRNA were prepared by diluting each probe to 1800 nM in Optimem base media. Target 1 probes (T1-1, T1-2, T1-3, and T1-5) were also pooled in Optimem base media to a concentration of 1800 nM for each probe. The 1800 nM working solutions were then serial diluted in Optimem base media to 600, 200, 66.67, 22.22, 7.41, and 2.47 nM.

Additionally, working solutions and serial dilutions of DsiRNA specific for Target 3 (T3-1, T3-2, T3-3, T3-4, and T3-6) were prepared individually and as a pool at a concentration of 1800 nM by the method described above as a counter-screen for Target 1 in SCC25 cell viability.

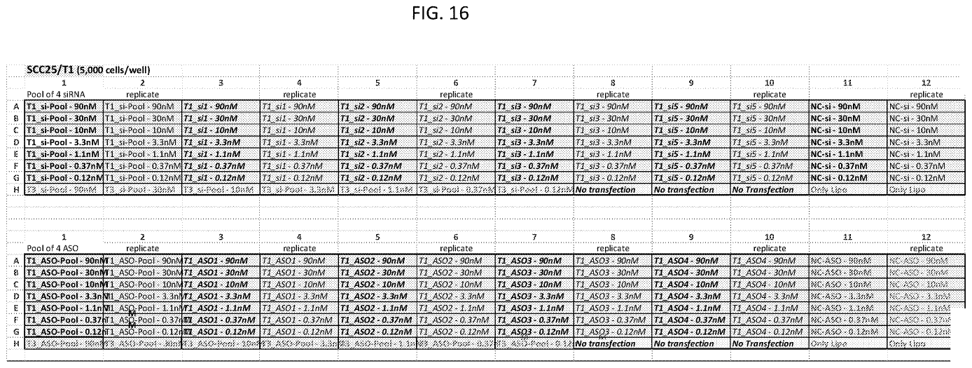

LIPOFECTAMINE™/DsiRNA transfection solutions were prepared at 10× final concentration by first diluting LIPOFECTAMINE™ 2000 25-fold in OptiMEM base medium. Working solutions of DsiRNA probes or pool were mixed with an equal volume of diluted LIPOFECTAMINE™ 2000 and incubated at ambient temperature for 20 minutes. 15 UL of each Transfection solution was then plated into individual wells of five 96-well tissue culture plates according to platemap. (Plates correspond to post transfection timepoints of 0, 24, 48, 96, and 144 hours.) SCC25 cells were diluted to 33,333 cells/mL in OptiMEM Reduced Serum medium containing 5% FBS and subsequently added to all wells of the five plates containing DsiRNA transfection solutions at 150 μL/well. Transfections were incubated for 48 hours at 37° C., 5% CO₂. SCC25 Platemap and the layout of DsiRNA and ASO probe pools for SCC25 transfection is shown in FIG. 16.

Cell Viability by Nuclear Staining and Laser Scanning Cytometry: After 48 hours, the transfection media was aspirated off, replaced with 200 μL of DMEM growth media and returned to a 37° C., 5% CO₂ incubator. At each timepoint, the plate was removed from the incubator, growth media removed and 100 μL of PBS containing the nuclear dyes Hoechst 33342 (8 UM) and Propidium Iodide (1.5 μM). Plates were placed back in the incubator for 30 minutes and then read on an SPT Labtech Mirrorball Laser Scanning Cytometer. Whole well fluorescent images at 405 nm excitation/460 nm emission (Hoechst 33342 live cell fluorescence) and 488 nm excitation/560 nm emission (Propidium Iodide dead/dying cell fluorescence) were collected and used to determine the growth rate and overall cell viability for each DsiRNA-treatment group at each timepoint. For the T=0 plate, the cells were washed with PBS after data collection followed by removal of PBS and subsequent addition of Qiagen RLT reagent to lyse and stabilize RNA for later analysis. Lysed cell plates were placed at −80° C.

### Target 1 Knockdown of SCC25 Growth and Viability by ASO

Materials. SCC25 (CRL-1628), Dulbecco's modified Eagle's Medium: Ham's F12 medium (DMEM/F12; 30-2006), Fetal bovine serum (FBS; 30-2020) from ATCC, human insulin (12585-014) and Trypsin-EDTA 0.25% (25200-056) from Gibco, hydrocortisone (A16292-03), LIPOFECTAMINE™ 2000 Reagent (11668027), OptiMEM Reduced Serum Medium (OptiMEM, 31985088), SuperScript IV VILO Master Mix (11766050), Hoechst 33342 (H1399), Propidium Iodide (J66584-AB) and custom Taqman assays for Target 1 and Target 3 from Thermo Fisher Scientific, Antisense Oligonucleotides (ASOs) for Target 1, Target 3, and Negative Control ASO from Integrated DNA Technologies, RNeasy Plus Mini Kit (74134) from Qiagen and LUNA® Universal Probe qPCR Master Mix (M3004) from New England BioLabs were utilized in all experiments.

Cell Culture: SCC25 cells were cultured and maintained in DMEM/F12 medium with 10% FBS and 400 ng/ml hydrocortisone with subculturing at a split ratio of 1:10 every 4 days.

ASO Transfection: ASOs stock solutions for Target 1 probes were resuspended according to manufacturer's protocol; T1-ASO-1, T1-ASO-2, T1-ASO-3, and T1-ASO-4. Briefly, individual Target ASOs were resuspended in nuclease-free water to 75,000 nM. Negative Control ASO were solubilized to 25,000 nM in nuclease-free water.

Working solutions of individual ASOs and negative control ASO were prepared by diluting each probe to 1800 nM in Optimem base media. Target 1 probes (T1-ASO-1, T1-ASO-2, T1-ASO-3, and T1-ASO-4) were also pooled in Optimem base media to a concentration of 1800 nM for each probe. The 1800 nM working solutions were then serial diluted in Optimem base media to 600, 200, 66.67, 22.22, 7.41, and 2.47 nM.

Additionally, working solutions and serial dilutions of ASOs specific for Target 3 (T3-ASO-1, T3-ASO-2, and T3-ASO-3) were prepared individually and as a pool at a concentration of 1800 nM by the method described above as a counter-screen for Target 1 in SCC25 cell viability.

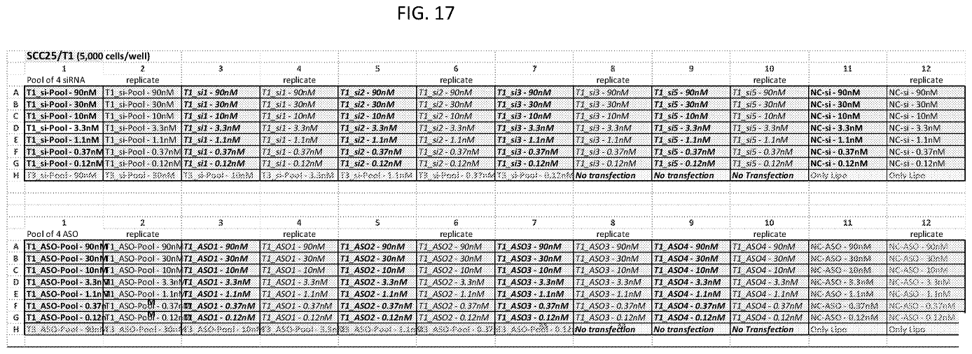

LIPOFECTAMINE™/ASO transfection solutions were prepared at 10× final concentration by first diluting LIPOFECTAMINE™ 2000 25-fold in OptiMEM base medium. Working solutions of ASO probes or pool were mixed with an equal volume of diluted LIPOFECTAMINE™ 2000 and incubated at ambient temperature for 20 minutes. 15 μL of each Transfection solution was then plated into individual wells of five 96-well tissue culture plates according to platemap (Plates correspond to post transfection timepoints of 0, 24, 48, 96, and 144 hours). SCC25 cells were diluted to 33,333 cells/mL in OptiMEM Reduced Serum medium containing 5% FBS and subsequently added to all wells of the five plates containing DsiRNA transfection solutions at 150 μL/well. Transfections were incubated for 48 hours at 37° C., 5% CO₂. SCC25 Platemap and the layout of DsiRNA and ASO probe pools for SCC25 transfection is shown in FIG. 17

Cell Viability by Nuclear Staining and Laser Scanning Cytometry: After 48 hours, the transfection media was aspirated off, replaced with 200 μL of DMEM growth media and returned to a 37° C., 5% CO₂ incubator. At each timepoint, the plate was removed from the incubator, growth media removed and 100 μL of PBS containing the nuclear dyes Hoechst 33342 (8 UM) and Propidium Iodide (1.5 μM). Plates were placed back in the incubator for 30 minutes and then read on an SPT Labtech Mirrorball Laser Scanning Cytometer. Whole well fluorescent images at 405 nm excitation/460 nm emission (Hoechst 33342 live cell fluorescence) and 488 nm excitation/560 nm emission (Propidium Iodide dead/dying cell fluorescence) were collected and used to determine the growth rate and overall cell viability for each ASO-treatment group at each timepoint. For the T=0 plate, the cells were washed with PBS after data collection followed by removal of PBS and subsequent addition of Qiagen RLT reagent to lyse and stabilize RNA for later analysis. Lysed cell plates were placed at −80° C.

### Target 3 Knockdown of Hs578T Growth and Viability by ASO

Materials. Hs578T (HTB-126), Dulbecco's Modified Eagle's Medium (DMEM; 30-2002), Fetal bovine serum (FBS; 30-2020) from ATCC, human insulin (12585-014) and Trypsin-EDTA 0.25% (25200-056) from Gibco, hydrocortisone (A16292-03), LIPOFECTAMINE™ 2000 Reagent (11668027), OptiMEM Reduced Serum Medium (OptiMEM, 31985088), SuperScript IV VILO Master Mix (11766050), Hoechst 33342 (H1399), Propidium Iodide (J66584-AB) and custom Taqman assays for Target 1 and Target 3 from Thermo Fisher Scientific, Antisense Oligonucleotides (ASOs) for Target 1, Target 3, and Negative Control ASO from Integrated DNA Technologies, RNeasy Plus Mini Kit (74134) from Qiagen and LUNA® Universal Probe qPCR Master Mix (M3004) from New England BioLabs were utilized in all experiments.

Cell Culture: Hs578T cells were cultured and maintained in DMEM medium with 10% FBS and 0.01 mg/ml insulin with subculturing at a split ratio of 1:5 every 4 days.

DsiRNA Transfection: ASO stock solutions for Target 3 probes were resuspended according to manufacturer's protocol; T3-ASO-1, T3-ASO-2, and T3-ASO-3. Briefly, individual Target ASOs were resuspended in nuclease-free water to 75,000 nM. Negative Control ASO was solubilized to 25,000 nM in nuclease-free water.

Working solutions of individual ASOs and negative control ASO were prepared by diluting each probe to 1800 nM in Optimem base media. Target 3 probes (T3-ASO-1, T3-ASO-2, and T3-ASO-3) were also pooled in Optimem base media to a concentration of 1800 nM for each probe. The 1800 nM working solutions were then serial diluted in Optimem base media to 600, 200, 66.67, 22.22, 7.41, and 2.47 nM.

Additionally, working solutions and serial dilutions of ASOs specific for Target 1 (T1-ASO-1, T1-ASO-2, T1-ASO-3, and T1-ASO-4) were prepared individually and as a pool at a concentration of 1800 nM by the method described above as a counter-screen for Target 3 in Hs578T cell viability.

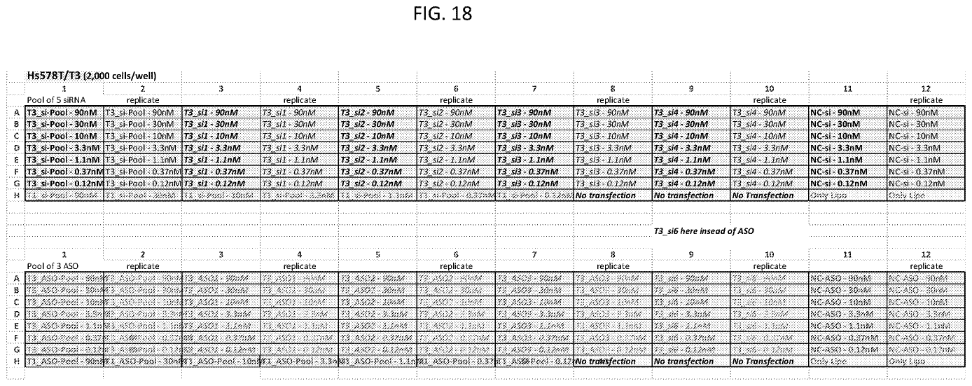

LIPOFECTAMINE™/ASO transfection solutions were prepared at 10× final concentration by first diluting LIPOFECTAMINE™ 2000 25-fold in OptiMEM base medium. Working solutions of ASO probes or pool were mixed with an equal volume of diluted LIPOFECTAMINE™ 2000 and incubated at ambient temperature for 20 minutes. 15 μL of each transfection solution was then plated into individual wells of five 96-well tissue culture plates according to platemap. (Plates correspond to post transfection timepoints of 0, 24, 48, 72, and 96 hours.) Hs578T cells were diluted to 13,333 cells/mL in OptiMEM Reduced Serum medium containing 5% FBS and subsequently added to all wells of the five plates containing DsiRNA transfection solutions at 150 μL/well. Transfections were incubated for 48 hours at 37° C., 5% CO₂. Hs578T Platemap and the layout of DsiRNA and ASO probe pools for Hs578T transfection is shown in FIG. 18.

Cell Viability by Nuclear Staining and Laser Scanning Cytometry: After 48 hours, the transfection media was aspirated off, replaced with 200 μL of DMEM growth media and returned to a 37° C., 5% CO₂ incubator. At each timepoint, the plate was removed from the incubator, growth media removed and 100 μL of PBS containing the nuclear dyes Hoechst 33342 (8 μM) and Propidium Iodide (1.5 μM). Plates were placed back in the incubator for 30 minutes and then read on an SPT Labtech Mirrorball Laser Scanning Cytometer. Whole well fluorescent images at 405 nm excitation/460 nm emission (Hoechst 33342 live cell fluorescence) and 488 nm excitation/560 nm emission (Propidium Iodide dead/dying cell fluorescence) were collected and used to determine the growth rate and overall cell viability for each ASO-treatment group at each timepoint. For the T=0 plate, the cells were washed with PBS after data collection followed by removal of PBS and subsequent addition of Qiagen RLT reagent to lyse and stabilize RNA for later analysis. Lysed cell plates were placed at −80° C.

### Target 3 Knockdown of Hs578T Growth and Viability by DsiRNA

Materials. Hs578T (HTB-126), Dulbecco's Modified Eagle's Medium (DMEM; 30-2002), Fetal bovine serum (FBS; 30-2020) from ATCC, human insulin (12585-014) and Trypsin-EDTA 0.25% (25200-056) from Gibco, hydrocortisone (A16292-03), LIPOFECTAMINE™ 2000 Reagent (11668027), OptiMEM Reduced Serum Medium (OptiMEM, 31985088), SuperScript IV VILO Master Mix (11766050), Hoechst 33342 (H1399), Propidium Iodide (J66584-AB) and custom Taqman assays for Target 1 and Target 3 from Thermo Fisher Scientific, Dicer-Substrate Short Interfering RNAs (DsiRNAs) for Target 1, Target 3, HPRT-S1 (51-01-08-02), and Negative Control (51-01-14-03), Human HPRT qPCR Assay (51-01-08-04) and Human GAPDH qPCR Assay (Hs.PT.39a.22214836) from Integrated DNA Technologies, RNeasy Plus Mini Kit (74134) from Qiagen and LUNA® Universal Probe qPCR Master Mix (M3004) from New England BioLabs were utilized in all experiments.

Cell Culture: Hs578T cells were cultured and maintained in DMEM medium with 10% FBS and 0.01 mg/ml insulin with subculturing at a split ratio of 1:5 every 4 days. SCC25 cells were cultured and maintained in DMEM/F12 medium with 10% FBS and 400 ng/ml hydrocortisone with subculturing at a split ratio of 1:10 every 4 days.

DsiRNA Transfection: DsiRNAs stock solutions for Target 3 probes were resuspended according to manufacturer's protocol; T3-1, T3-2, T3-3, T3-4, and T3-6. Briefly, individual Target DsiRNAs were resuspended in nuclease-free water to 75,000 nM. Negative Control DsiRNA were solubilized to 25,000 nM in nuclease-free water.

Working solutions of individual DsiRNAs and negative control DsiRNA were prepared by diluting each probe to 1800 nM in Optimem base media. Target 3 probes (T3-1, T3-2, T3-3, T3-4, and T3-6) were also pooled in Optimem base media to a concentration of 1800 nM for each probe. The 1800 nM working solutions were then serial diluted in Optimem base media to 600, 200, 66.67, 22.22, 7.41, and 2.47 nM.

Additionally, working solutions and serial dilutions of DsiRNA specific for Target 1 (T1-1, T1-2, T1-3, and T1-5) were prepared individually and as a pool at a concentration of 1800 nM by the method described above as a counter-screen for Target 3 in Hs578T cell viability.

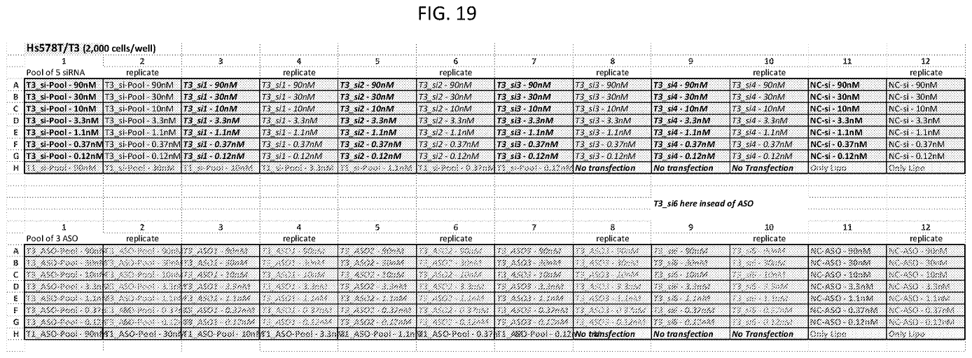

LIPOFECTAMINE™/DsiRNA transfection solutions were prepared at 10× final concentration by first diluting LIPOFECTAMINE™ 2000 25-fold in OptiMEM base medium Working solutions DsiRNA probes or pool were mixed with an equal volume of diluted LIPOFECTAMINE™ 2000 and incubated at ambient temperature for 20 minutes. 15 μL of each Transfection solution was then plated into individual wells of five 96-well tissue culture plates according to platemap (Plates correspond to post transfection timepoints of 0, 24, 48, 72, and 96 hours). Hs578T cells were diluted to 13,333 cells/mL in OptiMEM Reduced Serum medium containing 5% FBS and subsequently added to all wells of the five plates containing DsiRNA transfection solutions at 150 μL/well. Transfections were incubated for 48 hours at 37° C., 5% CO₂. Hs578T Platemap and the layout of DsiRNA and ASO probe pools for Hs578T transfection is shown in FIG. 19.

Cell Viability by Nuclear Staining and Laser Scanning Cytometry: After 48 hours, the transfection media was aspirated off, replaced with 200 μL of DMEM growth media and returned to a 37° C., 5% CO₂ incubator. At each timepoint, the plate was removed from the incubator, growth media removed and 100 μL of PBS containing the nuclear dyes Hoechst 33342 (8 μM) and Propidium Iodide (1.5 μM). Plates were placed back in the incubator for 30 minutes and then read on an SPT Labtech Mirrorball Laser Scanning Cytometer. Whole well fluorescent images at 405 nm excitation/460 nm emission (Hoechst 33342 live cell fluorescence) and 488 nm excitation/560 nm emission (Propidium Iodide dead/dying cell fluorescence) were collected and used to determine the growth rate and overall cell viability for each DsiRNA-treatment group at each timepoint. For the T=0 plate, the cells were washed with PBS after data collection followed by removal of PBS and subsequent addition of Qiagen RLT reagent to lyse and stabilize RNA for later analysis. Lysed cell plates were placed at −80° C.

### Cell Transfection and Immunofluorescence Characterization:

Materials: HEK293T (CRL-3216), Dulbecco's Modified Eagle's Medium (DMEM; 30-2002), Fetal bovine serum (FBS; 30-2020) from ATCC and Trypsin-EDTA 0.25% (25200-056) from Gibco, LIPOFECTAMINE™ 2000 Reagent (11668027), OptiMEM Reduced Serum Medium (OptiMEM, 31985088), Hoechst 33342 (H1399), Propidium Iodide (J66584-AB) from Thermo Fisher Scientific, AF594-labelled Anti-FLAG antibody (#20861S) and 16% formaldehyde (#18814) from Cell Signaling Technology, plasmids containing sequences from four targets in a pCMV-3Tag-1a backbone and GFP-FLAG plasmid (AG13105-CF) from Sino Biologic.

Cell Culture: HEK293T cells were cultured and maintained in DMEM medium with 10% FBS with subculturing at a split ratio of 1:8 every 3 days.

Plasmid Transfection: Plasmid stock solutions were resuspended to 1000 ng/uL in TE buffer. Working solutions of individual plasmids and GFP control plasmid were prepared by diluting each probe to 20 ng/uL in Optimem base media. LIPOFECTAMINE™/plasmid transfection solutions were prepared at 10× final concentration by first diluting LIPOFECTAMINE™ 2000 25-fold in OptiMEM base medium. Working plasmid solutions were mixed with an equal volume of diluted LIPOFECTAMINE™ 2000 and incubated at ambient temperature for 5 minutes. 100 μL of HEK293T cells in OptiMEM Reduced Serum medium containing 5% FBS (20,000 cells/mL) were seeded into 96-well tissue culture plates or poly-d-lysine-coated tissue culture plates and subsequently treated with 12.5 μL or 25 μL of each plasmid working solution. Transfections were incubated for 48 hours at 37° C., 5% CO₂.

Overexpression Analysis by GFP Fluorescence and FLAG-tag Immunofluorescence: During transfection, efficiency was monitored by measuring GFP Fluorescence on an epifluorescence microscope (10×@ 488 nm excitation/530 nm emission). After 48 hours, the transfection media was aspirated off, replaced with 200 μL of PBS to measure final GFP levels. After GFP fluorescence detection, buffer was removed, and cells fixed with 25 μL of 4% formaldehyde diluted in 1×PBS. After fixation at 30 min at room temperature, the fixative was removed, and plate washed 3 times with 150 μL PBS per well. 100 μL of Blocking Buffer (1×PBS/1% BSA/0.3% Triton™ X-100) was added and incubated for 60 min at room temperature. After blocking, 50 μL of a 1:50 dilution of anti-FLAG/AF5894 in antibody dilution buffer (1×PBS/1% BSA/0.3% Triton™ X-100) was added and incubated overnight at 4° C. Plates were then washed 3 times with PBS and imaged on an epifluorescence microscope at 10× magnification using 488 nm excitation/530 nm emission for GFP Fluorescence and 530 nm excitation/590 nm emission for FLAG-Tag Fluorescence.

### Target 2 and Target 5 Sequences

Target 2 (siRNA targets are bolded and underlined)
(SEQ ID NO: 23)
ATGGACTTTTCCATCTGCATCAGTAACATCACCCCAGCAGATGCCGGCAC
CTACTACTGTGTGAAGTTCCAGAAAGGGAGCCCTGACGTGGAGTTGAAGT
CTGGAGCAGGCACTGAGCTGTCTGTGCGTGCCAAACCCTCTGCCCCCGTG
GTATCGGGCCCCGCAGCGAGGGCCACACCTGACCACACAGTGAGCTTCAC
CTGCGAGTCTCATGGCTTCTCACCCAGAGACATCAGCCTGAAATGGTTCA
AAAATGGGAATCAGCTCTCAGACTTCCAGACCAACGTGGACCCCGCAAGA
GAGAGCGTGTCCTACAGCATCCACAGCACAGCCAATGTGGTGCTGACCCG
CGGGGACATTCACTCTCAAGTCATCTGCGAGGTGGCCCACGTCACCTTGC
GGGGGGACTCTTTTCGTGGGACTGCCAACTTGTCTGAGACTATCCAAGTT
CCACCCACCTTGGAGGTTACTCAACAGCCCATGAGGGCAGAGAACCAGGT
GAATATCACCTGCCAGGTGACGAAATTCTACCCCCAGAGACTACAGTTGA
CCTGGTTGGAGAACGGCAATGTGTCCCGGACAGAAACGGCCTCAACTCTT
ACAGAGAACAAGGATGGCACCTACAACTGGATGAGCTGGCTCCTGGTGAA
TGTATCTGCCCACAGGGATGATGTGAAGCTCACCTGCCAGGTGGAGCATG
ACGGGCAGTCAGCGGTCAGCAAAAGCCATGACCTGAAGGTCTCAGCCCAC
CTGAAGGAGCAGAGCTCAAATACCGCCGCTGAGAACACTGGACCTAATGA
ACAGAACATCTATATTGTGGTGGGCGTGGTGTGCACCTTGCTGGTGGCCC
TACTGATGGAGGCTCTCTACCTCGTCCGAATCAGACAGAAGAAAGCCCAG
GGCTCCACTTCTTCTACAAGGTTGCATGAACCCGAGAAGAATGCCAGAAA
AATAACCCAGGACACAAATGATATCACATATGCGGACCTGAACCTGCCCA
AGGGGAAGAAGCCTGCTCCCCGGGCCGCGGAGCCCAACAACCACACAGAG
TATGCCAGCATTCAGACCAGCCTGCAGCCTGCGTCGGAGGACACCCTCAC
CTATGCTGACCTGGACATGGTGCACCTCAACCGGACCCCCAAGCAGCTGG
CCCCCAAGCCCGAGCTGTCCTTCTCAGAGTATGCCAGCATCCAGGTCCCG
AGGAAGTGAATGGGACCGTGGTTTGCTCTAGCACCCATCTCCATGCTCTT
CCTTGTCCCACAAGGAGCCGCCATGATGAGCACAGCCAGCCCAGTTCCCG
GAGGGCTGGGGCGGTGCAGGCTCTGGGACCCAGGGGCCAGGGTGGCTCTT
CTCTCCCCACCCCTCCTTGGCTCTCCAGCAGCCACGGCCCCCTCTCCCCA
CATTGCCACACACCTGGAGGCTGACGTTGCC
Target 5 (siRNA targets are bolded and underlined)
(SEQ ID NO: 24)
ATGGGGATTCCAGGCCTGGAGCACCTGCACATCTGGATCTCCATCCCCTT
CTCAGCATATACACTGGCCCTGCTTGGAAACTGCACCCTCCTTCTCATCA
TCCAGGCTGATGCAGCCCTCCATGAGCCCATATACCTCTTTCTGGCCATG
TTGGCAGCCATCGACCTGGTCCTTTCCTCCTCAGCATTGCCCAAAATGCT
TGCCATATTCTGGTTCAGGGATCGGGAGATCAACTTTTTTGCCTGTCTGG
TCCAGATGTTCTTCCTTCACTCCTTCTCCATCATGGAGTCAGCAGTGCTG
CTGGCCATGGCCTTTGACCGCTATGTGGCCATCTGCAAGCCACTGCACTA
CACCACGGTCCTGACTGGGTCCCTCATCACCAAGATTGGCATGGCTGCTG
TGGCCCGGGCTGTGACACTAATGACTCCACTCCCCTTCCTGCTGAGATGT
TTCCACTACTGCCGAGGCCCAGTGATTGCCCGCTGCTACTGTGAACACAT
GGCTGTGGTCAGGCTGGCTGTGGGAACACTAGCTTCAACAATATCTATGG
CATTGCTGTGGCCATGTTTATTGGAGTGTTGGATCTATTCTTTATCATCC
TATCTTATATCTTTATCCTTCAGGCAGTTCTACAACTCTCCTCTCAGGAG
GCCCGCTACAAAGCATTTGGGACATGTGTCTCTCAC

TABLE 5
Target 2 siRNA sequences

| Target | SEQ ID NO | Sequence 1 | SEQ ID NO: | Sequence 2 | SEQ ID NO: |
|---|---|---|---|---|---|
| CAGCAAAAGCCA | SEQ ID NO: 25 | rArCrArUrUrCrAr | SEQ ID NO: 26 | rUrCrGrCrArGrA | SEQID NO: 27 |
| TGACCTGAAGGT | | CrUrCrUrCrArAr | | rUrGrArCrUrUrG | |
| C | | GrUrCrArUrCrUr | | rArGrArGrUrGrA | |
| | | GrCGA | | rArUrGrUrCrC | |
| AACTTGTCTGAG | SEQ ID NO: 28 | rCrArGrCrArArAr | SEQ ID NO: 29 | rGrArCrCrUrUrC | SEQ ID NO: 30 |
| ACTATCCAAGTT | | ArGrCrCrArUrGr | | rArGrGrUrCrArU | |
| C | | ArCrCrUrGrArAr | | rGrGrCrUrUrUrU | |
| | | GrGTC | | rGrCrUrGrArC | |
| ACATTCACTCTC | SEQ ID NO: 31 | rArArCrUrUrGrUr | SEQ ID NO: 32 | rGrArArCrUrUrG | SEQ ID NO: 33 |
| AAGTCATCTGCG | | CrUrGrArGrArCr | | rGrArUrArGrUrC | |
| A | | UrArUrCrCrArAr | | rUrCrArGrArCrAr | |
| | | GrUTC | | ArGrUrUrGrG | |

TABLE 6
Target 5 siRNA sequences

| Target | SEQ ID NO | Sequence 1 | SEQ ID NO: | Sequence 2 | SEQ ID NO: |
|---|---|---|---|---|---|
| CTACAAAGCATT | SEQ ID NO: 34 | rCrUrArCrArArAr | SEQ ID NO: 35 | rGrArCrArCrArUr | SEQ ID NO: 36 |
| TGGGACATGTGT | | GrCrArUrUrUrGr | | GrUrCrCrCrArAr | |
| C | | GrGrArCrArUrGr | | ArUrGrCrUrUrUr | |
| | | UrGTC | | GrUrArGrCrG | |
| CTTCAACAATAT | SEQ ID NO: 37 | rCrUrUrCrArArCr | SEQ ID NO: 38 | rArGrCrArArUrGr | SEQ ID NO: 39 |
| CTATGGCATTGC | | ArArUrArUrCrUr | | CrCrArUrArGrAr | |
| T | | ArUrGrGrCrArUr | | UrArUrUrGrUrUr | |
| | | UrGCT | | GrArArGrCrU | |
| CCTTCCTGCTGA | SEQ ID NO: 40 | rCrCrUrUrCrCrU | SEQ ID NO: 41 | rUrArGrUrGrGrA | SEQ ID NO: 42 |
| GATGTTTCCACT | | rGrCrUrGrArGrA | | rArArCrArUrCrUr | |
| A | | rUrGrUrUrUrCrC | | CrArGrCrArGrGr | |
| | | rArCTA | | ArArGrGrGrG | |

TABLE 7
Taqman assay sequences

| | | | | Re- | Reporter | Forward | SEQ | Reverse | SEQ | Reporter | SEQ |
|---|---|---|---|---|---|---|---|---|---|---|---|
| Assay | | Tar- | | porter | 1 | Primer | ID | Primer | ID | 1 | ID |
| ID | Vendor | get | Catalog | 1 Dye | Quencher | Sequence | NO: | Sequence | NO: | Sequence | NO: |
| APWC3ZF | Thermo | 1 | T1- | FAM | NFQ | CGCCCA | 43 | CGCGCCATT | 44 | ACAAGAA | 45 |
| | | | 455557 | | | GCCCCT | | GCATTCCA | | CAAAACT | |
| | | | | | | TTCATT | | | | CCATATC | |
| | | | | | | | | | | AA | |
| APXGXKD | Thermo | 2 | T2- | FAM | NFQ | CCTGCC | 47 | CGGGACACA | 48 | CCAACCA | 49 |
| | | | 448467 | | | AGGTGA | | TTGCCGTTC | | GGTCAAC | |
| | | | | | | CGAAAT | | T | | TGTA | |
| | | | | | | TCTAC | | | | | |
| APU7AFH | Thermo | 3 | T3- | FAM | NFQ | CTCAGT | 51 | CATCACACC | 52 | CCATGAG | 53 |
| | | | 383686 | | | GCCCAG | | TGAACCCTC | | GACAGAT | |
| | | | | | | GAAGAT | | TT | | TAC | |
| | | | | | | ACTC | | | | | |
| APT2FVK | Thermo | 5 | T5- | FAM | NFQ | CCCGG | 55 | CGGCAGTAG | 56 | ACTCCAC | 57 |
| | | | 531655 | | | GCTGTG | | TGGAAACAT | | TCCCCTT | |
| | | | | | | ACACTA | | CTCA | | CCTG | |
| | | | | | | ATG | | | | | |
| APT2FVK | IDT | GAPDH | Hs.PT. | SUN | IBQ | ACATCG | 59 | TGTAGTTGA | 60 | AAGGTCG | 61 |
| | | | 39a. | | | CTCAGA | | GGTCAATGA | | GAGTCAA | |
| | | | 22214836 | | | CACCAT | | AGGG | | CGGATTT | |
| | | | | | | G | | | | GGTC | |
| | IDT | HPRT1 | Hs.PT. | FAM | IBQ | TTGTTG | 62 | GCGATGTCA | 63 | AGCCTAA | 64 |
| | | | 58v. | | | TAGGAT | | ATAGGACTC | | GATGAGA | |
| | | | 45621572 | | | ATGCCC | | CAG | | GTTCAAG | |
| | | | | | | TTGA | | | | TTGAGTT | |
| | | | | | | | | | | TGG | |
| | | | | | | | | | | | SEQ |
| | | | | | | | | | Assay | Context | ID |
| | | | | | | | | | ID | Sequence | NO: |
| | | | | | | | | | APWC3ZF | TTTGATA | 46 |
| | | | | | | | | | | TGGAGTT | |
| | | | | | | | | | | TTGTTCT | |
| | | | | | | | | | | TGTT | |
| | | | | | | | | | APXGXKD | CTACAGT | 50 |
| | | | | | | | | | | TGACCTG | |
| | | | | | | | | | | GTTGGA | |
| | | | | | | | | | | GAACG | |
| | | | | | | | | | APU7AFH | CTCAGTG | 54 |
| | | | | | | | | | | CCCAGG | |
| | | | | | | | | | | AAGATAC | |
| | | | | | | | | | | TCCTG | |
| | | | | | | | | | APT2FVK | CCCGGG | 58 |
| | | | | | | | | | | CTGTGAC | |
| | | | | | | | | | | ACTAATG | |
| | | | | | | | | | | ACTCC | |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1-59. (canceled)

60. A method of treating cancer in a subject comprising administering to the subject a polynucleotide that reduces expression of a gene ENST00000455557.2 having the sequence of SEQ ID NO: 1 to treat the cancer, wherein the polynucleotide targets one or more of nucleotides 130-597 of SEQ ID NO: 1.

61. The method of claim 60, wherein the cancer is head and neck cancer, bladder urothelial carcinoma, breast invasive carcinoma, cervical cancer, endocervical cancer, colon adenocarcinoma, esophageal carcinoma, kidney clear cell carcinoma, kidney papillary cell carcinoma, lung adenocarcinoma, lung squamous cell carcinoma, ovarian serous cystadenocarcinoma, pancreatic adenocarcinoma, rectum adenocarcinoma, skin cutaneous melanoma, stomach adenocarcinoma, uterine carcinosarcoma, or uterine corpus endometrioid carcinoma.

62. The method of claim 60, wherein the polynucleotide has a nucleobase sequence comprising a portion of at least 10 contiguous nucleobases having at least 80% complementarity to an equal length portion of nucleotides 130-597 of SEQ ID NO: 1.

63. The method of claim 60, wherein the oligonucleotide comprises a region complementary to at least 17 contiguous nucleotides of SEQ ID NO: 1.

64. The method of claim 63, wherein the oligonucleotide comprises a region complementary to at least 19 contiguous nucleotides of SEQ ID NO: 1.

65. The method of claim 60, wherein the method reduces expression of a polypeptide having the sequence of SEQ ID NO: 2.

66. The method of claim 60, wherein the polynucleotide comprises the sequence of any one of SEQ ID NOs: 5-13.

67. The method of claim 60, wherein the polynucleotide comprises a miRNA, an antisense polynucleotide, an shRNA, or an siRNA.

68. The method of claim 60, wherein the polynucleotide consists of 12 to 40 nucleobases.

69. The method of claim 60, wherein the polynucleotide consists of 18 to 22 nucleobases.

70. The method of claim 60, wherein the polynucleotide comprises at least one alternative internucleoside linkage.

71. The method of claim 60, wherein the polynucleotide comprises at least one alternative nucleobase.

72. The method of claim 60, wherein the polynucleotide comprises at least one alternative sugar moiety.

73. The method of claim 60, wherein the polynucleotide is encoded by a vector.

74. The method of claim 73, wherein the vector is a viral vector.

75. The method of claim 74, wherein viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

76. A method of treating cancer in a subject comprising administering to the subject a polynucleotide that reduces expression of a gene ENST00000383686.3 having the sequence of SEQ ID NO: 3 to treat the cancer, wherein the polynucleotide targets one or more of nucleotides 115-711 of SEQ ID NO: 3.

77. The method of claim 76, wherein the cancer is brain lower grade glioma, breast invasive carcinoma, diffuse large B cell lymphoma, esophageal carcinoma, kidney clear cell carcinoma, kidney papillary cell carcinoma, liver hepatocellular carcinoma, lung adenocarcinoma, lung squamous cell carcinoma, ovarian serous cystadenocarcinoma, pancreatic adenocarcinoma, prostate adenocarcinoma, skin cutaneous melanoma, stomach adenocarcinoma, testicular germ cell tumor, thymoma, thyroid carcinoma, uterine carcinosarcoma, or uterine corpus endometrioid carcinoma.

78. The method of claim 76, wherein the polynucleotide has a nucleobase sequence comprising a portion of at least 10 contiguous nucleobases having at least 80% complementarity to an equal length portion of nucleotides 115-711 of SEQ ID NO: 3.

79. The method of claim 76, wherein the oligonucleotide comprises a region complementary to at least 17 contiguous nucleotides of SEQ ID NO: 3.

80. The method of claim 76, wherein the method reduces expression of a polypeptide having the sequence of SEQ ID NO: 4.

81. The method of claim 76, wherein the polynucleotide comprises the sequence of any one of SEQ ID NOs: 14-22.

82. A polynucleotide that targets one or more of nucleotides 130-597 of SEQ ID NO: 1.

83. A polynucleotide that targets one or more of nucleotides 115-711 of SEQ ID NO: 3.
